(19) United States
(12) Patent Application Publication
(10) Pub. No.: US 2019/0031715 A1
(43) Pub. Date: Jan. 31, 2019

(54) PROCESS AND INTERMEDIATES FOR SYNTHESIS OF PEPTIDE COMPOUNDS

(71) Applicant: Naurex, Inc, Madison, NJ (US)

(72) Inventor: Saibaba Racha, Edison, NJ (US)

(21) Appl. No.: 16/046,351

(22) Filed: Jul. 26, 2018

Related U.S. Application Data
(60) Provisional application No. 62/538,117, filed on Jul. 28, 2017.

(51) Int. Cl. C07K 5/10; C07K 1/08; C07K 1/12

(52) U.S. Cl. CPC C07K 5/10; C07K 1/12; C07K 1/086; C07K 1/084

(57) ABSTRACT

Disclosed is a new process and intermediates for preparing dipyrrolidine peptide compounds such as, for example, rapastinel. Advantageously, the process may be industrially scalable and cost-effective and use less toxic reagents and/or solvents. Further, the process may be used to prepare peptide compounds having improved purity.

### BACKGROUND

An N-methyl-D-aspartate (NMDA) receptor is a postsynaptic, ionotropic receptor that is responsive to, inter alfa, the excitatory amino acids glutamate and glycine and the synthetic compound NMDA. The NMDA receptor (NMDAR) appears to control the flow of both divalent and monovalent ions into the postsynaptic neural cell through a receptor associated channel and has drawn particular interest since it appears to be involved in a broad spectrum of CNS disorders. The NMDAR has been implicated, for example, in neurodegenerative disorders including stroke-related brain cell death, convulsive disorders, and learning and memory. NMDAR also plays a central role in modulating normal synaptic transmission, synaptic plasticity, and excitotoxicity in the central nervous system. The NMDAR is further involved in Long-Term Potentiation (LTP), which is the persistent strengthening of neuronal connections that underlie learning and memory The NMDAR has been associated with other disorders ranging from hypoglycemia and cardiac arrest to epilepsy. In addition, there are preliminary reports indicating involvement of NMDA receptors in the chronic neurodegeneration of Huntington's, Parkinson's, and Alzheimer's diseases. Activation of the NMDA receptor has been shown to be responsible for post-stroke convulsions, and, in certain models of epilepsy, activation of the NMDA receptor has been shown to be necessary for the generation of seizures. In addition, certain properties of NMDA receptors suggest that they may be involved in the information-processing in the brain that underlies consciousness itself. Further, NMDA receptors have also been implicated in certain types of spatial learning.

In view of the association of NMDAR with various disorders and diseases, NMDA-modulating small molecule agonist and antagonist compounds have been developed for therapeutic use. NMDA receptor compounds may exert dual (agonist/antagonist) effect on the NMDA receptor through the allosteric sites. These compounds are typically termed “partial agonists”. In the presence of the principal site ligand, a partial agonist will displace some of the ligand and thus decrease Ca⁺⁺ flow through the receptor. In the absence of the principal site ligand or in the presence of a lowered level of the principal site ligand, the partial agonist acts to increase Ca⁺⁺ flow through the receptor channel.

Recently, an improved partial agonist of NMDAR with the following structure has been reported (rapastinel):

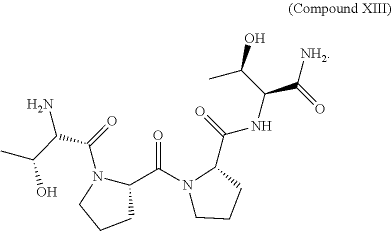

PCT/US2017/015851 describes a process for synthesis of peptide compounds, including rapastinel, the contents of which are incorporated herein by reference in its entirety. However, a need exists for improved rapastinel (GLYX-13) synthetic methods that, for example, minimize the use of costly and/or toxic reagents, eliminate cumbersome purification steps, are more efficient, result in higher purity rapastinel, and can be utilized in large-scale industrial production of rapastinel.

### SUMMARY

Disclosed is a new process for preparing dipyrrolidine peptide compounds such as, for example, rapastinel (GLYX-13). Advantageously, the process may be industrially scalable, produce higher yields and improved purity compared to those in the art, and be cost-effective and use less toxic reagents and/or solvents. Further, the process may be used to prepare peptide compounds having improved purity.

In one aspect, a process for synthesizing a dipyrrolidine peptide compound or a pharmaceutically acceptable salt, stereoisomer, metabolite, or hydrate thereof is provided. The process comprises the steps:
a) contacting a compound of Formula III:

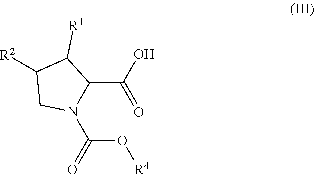

with an activating reagent and a compound of Formula II:

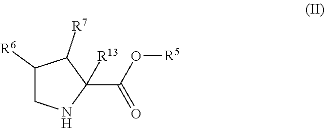

to produce a compound of Formula IV:

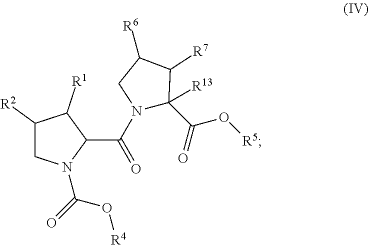

b) contacting the compound of Formula IV with a reagent capable of effecting hydrolysis to produce a compound of Formula V:

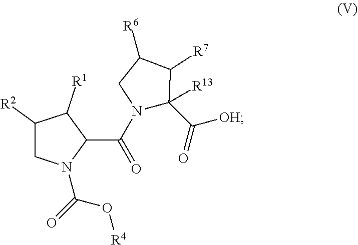

and
c) contacting the compound of Formula V with an activating reagent and a compound of Formula VIII:

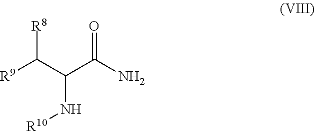

to produce a compound of Formula IX:

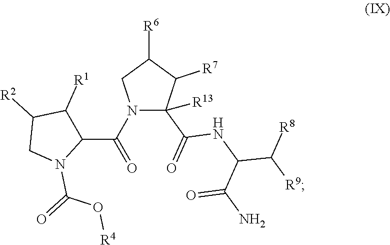

wherein:

R¹, R², R³, R⁴, R⁵, R⁶, R⁷, R⁸, R⁹, R¹⁰, R¹¹, R¹² and R¹³ are as defined below. In some embodiments, step (a) is carried out at a temperature between about −10° C. and about 10° C. In some embodiments, step (b) is carried out at a temperature between about 15° C. and about 30° C. In some embodiments, step (c) is carried out at a temperature between about 0° C. and about 30° C.

In some embodiments, the process further comprising the steps:
d) contacting the compound of Formula IX with a carbamate-cleaving reagent to produce a compound of Formula XI:

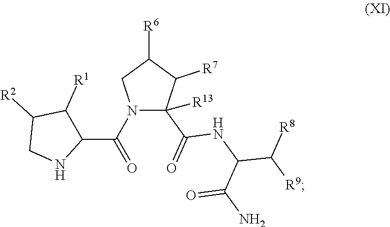

e) contacting a compound of Formula X:

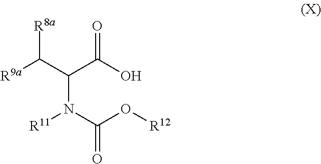

with an activating reagent and the compound of Formula XI to produce a compound of Formula XIIa:

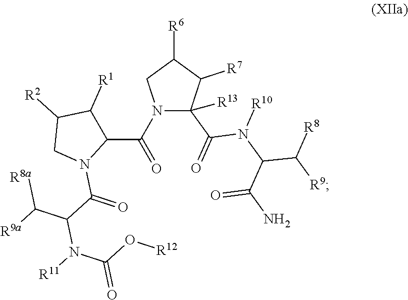

f) contacting the compound of Formula XIIa with a silyl ether cleaving reagent to produce a compound of Formula XII:

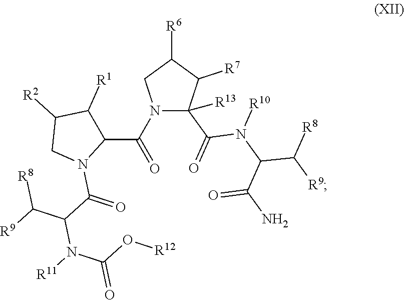

and
g) contacting the compound of Formula XII with a carbamate-cleaving reagent to produce a compound of Formula XIII:

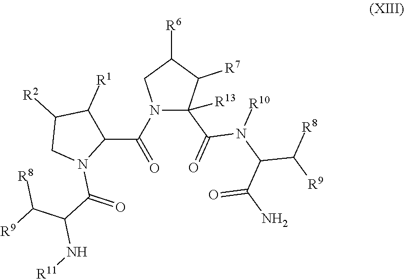

wherein R¹, R², R³, R⁴, R⁵, R⁶, R⁷, R⁸, R⁹, R^{9a}, R¹⁰, R¹¹, R¹², and R¹³ are as defined below. In some embodiments, step (d) is carried out at a temperature between about 15° C. and about 30° C. In some embodiments, step (e) is carried out at a temperature between about −10° C. and about 30° C. In some embodiments, step (f) is carried out at a temperature between about 15° C. and about 30° C.

In certain embodiments, the compound of Formula X is produced by contacting a compound of Formula VI:

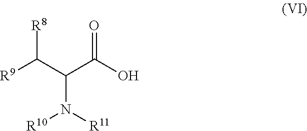

with an activated carbonyl compound followed by a silyl halide. In some embodiments, the compound of Formula VIII is produced by the steps:
g) contacting a compound represented by Formula VI:

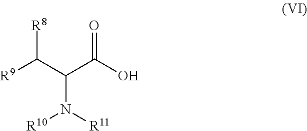

with an activating reagent to form a compound represented by Formula VII:

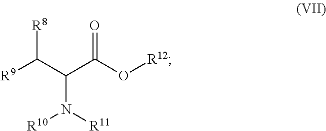

and
h) contacting the compound of Formula VII with an amine to produce the compound of Formula VIII. In some embodiments, step (g) is carried out at a temperature between about −10° C. and about 100° C. In some embodiments, step (h) is carried out at a temperature between about 15° C. and about 30° C.

In some cases, the compound of Formula II is produced by contacting a compound of Formula I:

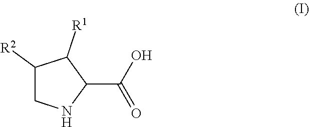

with an activating reagent and an alcohol. In some embodiments, producing the compound of Formula II is carried out at a temperature of between about 0° C. to about 100° C. In other embodiments, producing the compound of Formula II is carried out at a temperature of between about 0° C. to about 5° C.

In another aspect, a process for preparing a dipyrrolidine peptide compound or a pharmaceutically acceptable salt, stereoisomer, metabolite, or hydrate thereof is provided. The process comprises the steps:
a) contacting a compound of Formula IX:

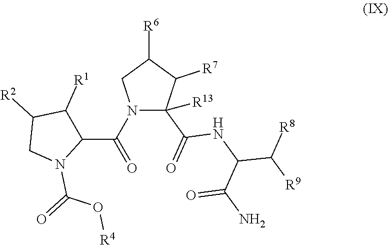

with a carbamate-cleaving reagent to produce a compound of Formula XI:

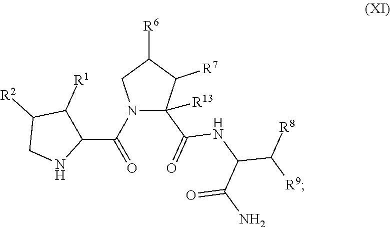

b) contacting a compound of Formula X:

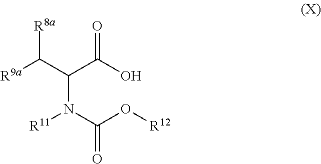

with an activating reagent and the compound of Formula XI in the presence of at least one solvent to produce a compound of Formula XIIa:

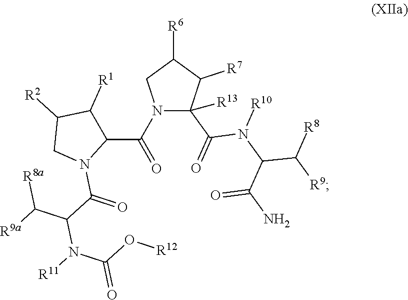

c) contacting the compound of Formula XIIa with a silyl ether cleaving reagent to produce a compound of Formula XII:

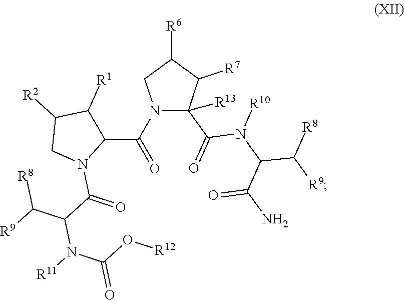

and
d) contacting the compound of Formula XII with a carbamate-cleaving reagent to produce a compound of Formula XIII:

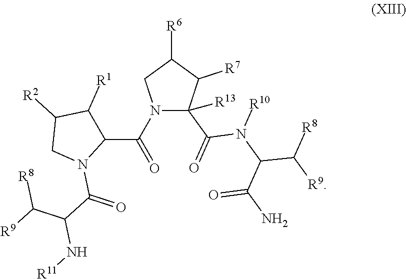

wherein:

R¹, R², R³, R⁴, R⁵, R⁶, R⁷, R⁸, R^{8a}, R⁹, R^{9a}, R¹⁰, R¹¹, R¹² and R¹³ are as defined below. In some embodiments, step (a) is carried out at a temperature between about 15° C. and about 30° C. In some instances, step (b) is carried out at a temperature of between about −10° C. to about 30° C. In some embodiments, step (c) is carried out at a temperature between about 15° C. and about 30° C.

In some embodiments, the compound of Formula IX is produced by:
d) contacting a compound of Formula III:

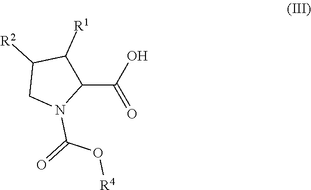

with an activating reagent and a compound of Formula II:

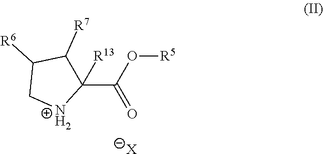

to produce a compound of Formula IV:

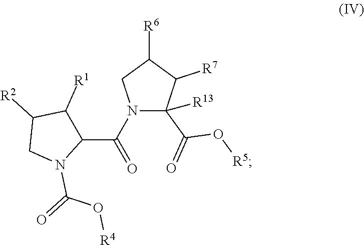

e) contacting the compound of Formula IV with a reagent capable of effecting hydrolysis to produce a compound of Formula V:

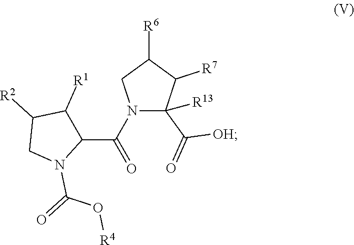

and
f) contacting the compound of Formula V with an activating reagent and a compound of Formula VIII:

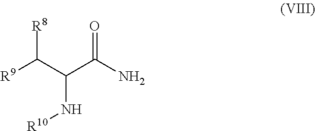

to produce a compound of Formula IX:

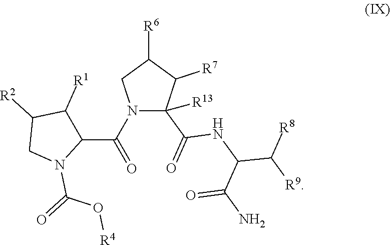

In some cases, step (e) is carried out at a temperature between about 15° C. and about 30° C. In some embodiments, step (f) is carried out at a temperature of between about 10° C. to about 30° C.

In some embodiments, the compound of Formula VIII is produced by the steps:
g) contacting a compound represented by Formula VI:

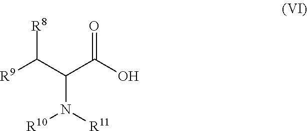

with an activating reagent to form a compound represented by Formula VII:

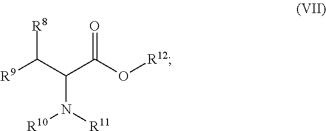

and
h) contacting the compound of Formula VII with an amine to produce the compound of Formula VIII. In some embodiments, step (g) is carried out at a temperature of between about 0° C. to 100° C. In some cases, step (h) is carried out at a temperature between about 15° C. to 30° C.

In some embodiments, the compound of Formula X is produced by contacting a compound of Formula VI:

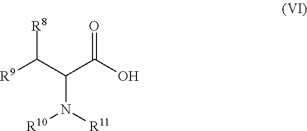

with an activated carbonyl compound. The process of claim 47 or 48, wherein producing the compound of Formula X is carried out at a temperature of between about 0° C. to about 30° C.

In some embodiments, the compound of Formula III is produced by contacting the compound of Formula II with an activated carbonyl reagent and a base. In some embodiments, the process further comprises contacting the compound of Formula VI with a base. In some instances, the base is NaHCO₃.

In some embodiments, the activating reagent comprises SOCl₂. In some instances, the alcohol is MeOH. In some embodiments, the activated carbonyl reagent is Cbz-Cl. In some cases, the base is a hydroxide salt. In some embodiments, the reagent capable of effecting hydrolysis comprises LiOH. For example, the reagent capable of effecting hydrolysis of the compound of Formula IV comprises LiOH. In some cases, the activating reagent comprises 1-ethyl-3-(3-dimethyllaminopropyl)carbodiimide. In some embodiments, the carbamate-cleaving reagent comprises palladium on carbon.

In some embodiments, the compound of Formula III is produced by contacting the compound of Formula I with an activating reagent and an alcohol to produce a reaction mixture comprising a compound of Formula II, and the reaction mixture is contacted with an activated carbonyl reagent and a base to produce the compound of Formula III.

In some embodiments, the compound of Formula VIII is produced by contacting the compound of Formula VI with an activating reagent and an alcohol to produce a reaction mixture comprising a compound of Formula VII, and the reaction mixture is contacted with an amine to produce the compound of Formula VIII. In some instances, the amine is NH₃.

In another aspect, a compound represented by the formula:

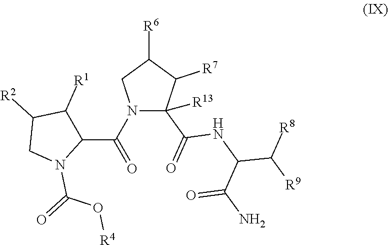

wherein:

R¹, R², R⁴, R⁶, R⁷, R⁸, R⁹, and R¹³ are as defined below is provided.

In some embodiments, one or more of R¹, R², R⁶, and R⁷ is hydrogen. In some cases, R⁸ is methyl. In certain embodiments, R⁹ is hydroxyl. In some instances, R⁴ is benzyl. In certain embodiments, R¹³ is hydrogen.

In some embodiments, a compound represented by the formula:

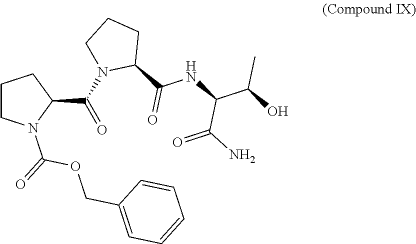

is provided.

In another aspect, a compound represented by the Formula X:

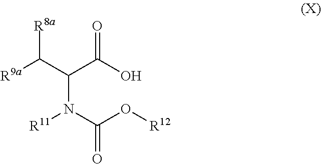

wherein:

R^{8a}, R^{9a}, R¹¹, and R¹² are as defined below is provided.

In some embodiments, R^{8a} is an alkylsilyl-O—, arylsilyl-O— or heteroarylsilyl-O—. In certain embodiments, R^{8a} is tertiary butyl dimethyl silyloxy. In some instances, R¹¹ is hydrogen. In some instances, R¹² is benzyl. In some embodiments, R⁹ is hydrogen.

In some embodiments, a compound represented by the formula:

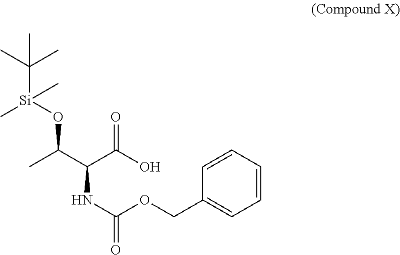 [SMILES: C[C@@H](O[Si](C)(C)C(C)(C)C)[C@H](NC(=O)OCc1ccccc1)C(=O)O]

is provided.

In another embodiment, a compound represented by the formula:

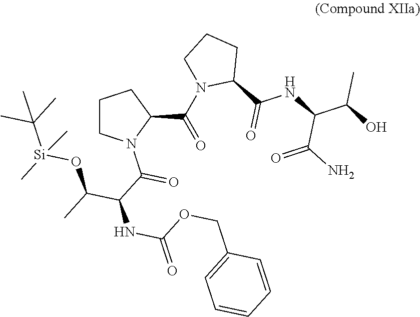 [SMILES: C[C@@H](O)[C@H](NC(=O)[C@@H]1CCCN1C(=O)[C@@H]1CCCN1C(=O)[C@@H](NC(=O)OCc1ccccc1)[C@@H](C)O[Si](C)(C)C(C)(C)C)C(N)=O]

is provided.

### BRIEF DESCRIPTION OF DRAWINGS

FIG. 1 is a schematic of a synthesis process for rapastinel according to an embodiment of this invention.

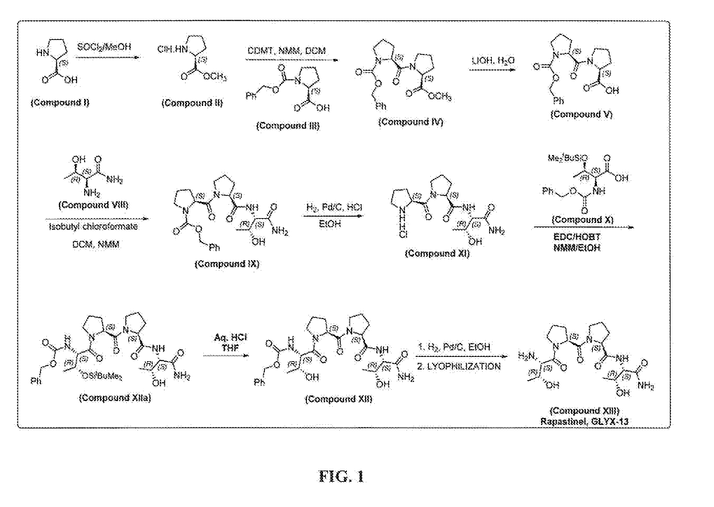

FIG. 2 is a schematic of synthesis processes for Compounds III, VIII and X according to another embodiment of this invention.

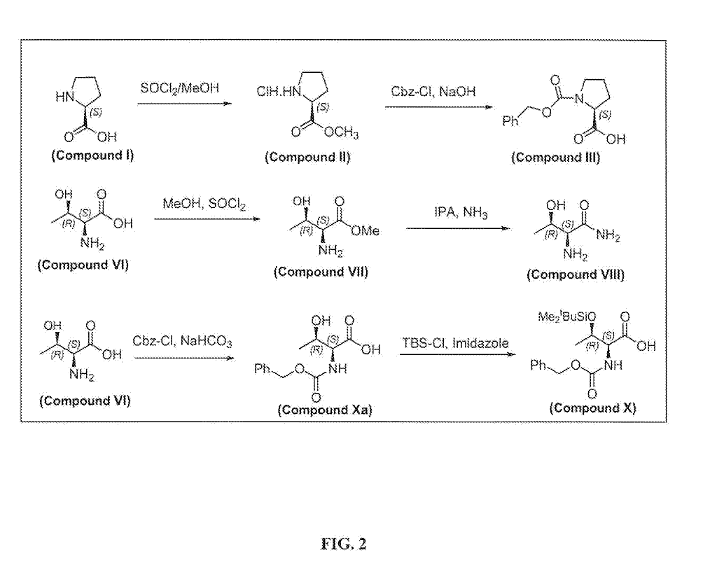

### DETAILED DESCRIPTION

Described herein is a new process for preparing dipyrrolidine peptide compounds. As a non-limiting example, the process may be used to prepare rapastinel or analogs or intermediates thereof. Advantageously, the process described herein may be used to prepare dipyrrolidine peptide compounds with higher purity and/or at less cost than known processes. Additionally, less toxic reagents and/or minimalist downstream processes may be used in contrast to known processes. Further, process may be scaled to produce industrial quantities of dipyrrolidine peptide compounds, e.g., greater than 1 kg of compound.

In some embodiments, the steps of the process may be carried out without using N-hydroxybenzotriazole (HOBT) and/or dichloromethane. This aspect may be advantageous since both HOBT and dichloromethane are costly raw materials, which increases the final process costs. Further, rapastinel is soluble in HOBT and the separation of this reaction mixture can be difficult. Consequently, the final purity of rapastinel may be compromised. Additionally, HOBT and dichloromethane are known to be toxic compounds, so their use introduces or increases the toxicity levels of the process. Of course, increased toxicity can result in increased process costs, for example, due to increased costs of handling toxic materials, increased waste disposal costs, and more expensive purification steps.

It will be appreciated by those of ordinary skill in the art that each of the embodiments contemplated herein may be utilized individually or combined in one or more manners different that the ones disclosed herein to produce an improved process for the production of dipyrrolidine peptide compounds. One skilled in the art will be able to select a suitable temperature and other such parameters in view of the reaction conditions being used in different embodiments.

Processes

In one embodiment, a process is provided for preparing a compound of Formula XIII (pharmaceutically acceptable salts, stereoisomers, metabolites, and hydrates thereof):

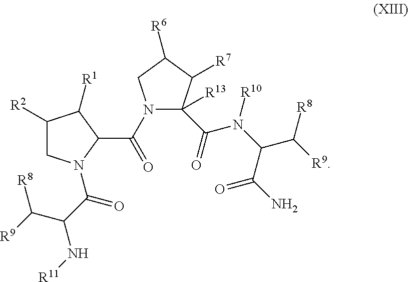

For example, a process is provided for preparing the compound rapastinel. A disclosed process may include:
a) contacting a compound of Formula III:

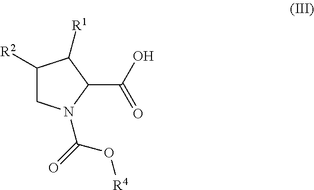

with an activating reagent and a compound of Formula II:

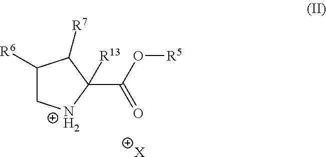

to produce a compound of Formula IV:

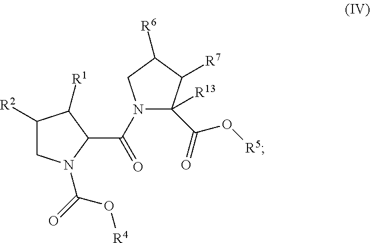

b) contacting the compound of Formula IV with a reagent capable of effecting hydrolysis to produce a compound of Formula V:

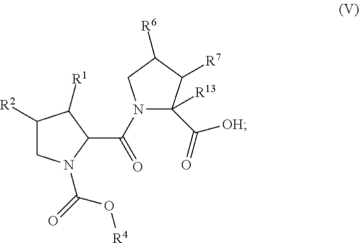

c) contacting the compound of Formula V with an activating reagent and a compound of Formula VIII:

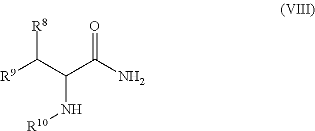

to produce a compound of Formula IX:

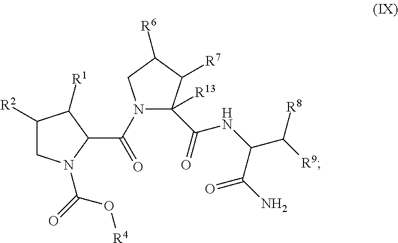

d) contacting the compound of Formula IX with a carbamate-cleaving reagent to produce a compound of Formula XI:

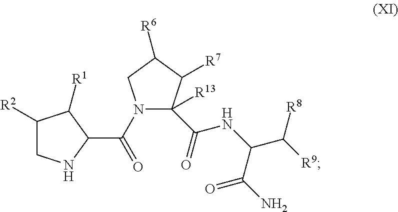

e) contacting a compound of Formula X:

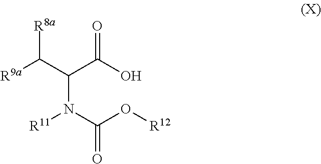

with an activating reagent and the compound of Formula XI to produce a compound of Formula XIIa:

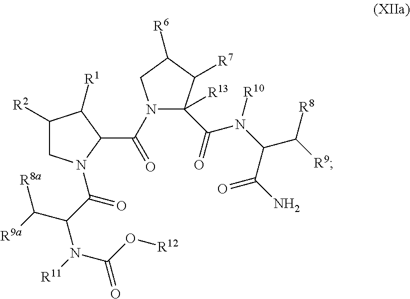

and
f) contacting the compound of Formula XIIa with a silyl ether cleaving reagent to produce a compound of Formula XII:

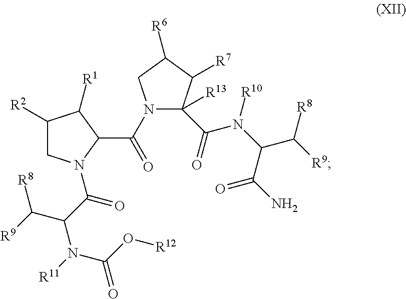

and
g) contacting the compound of Formula XII with a carbamate-cleaving reagent to produce a compound of Formula XIII:

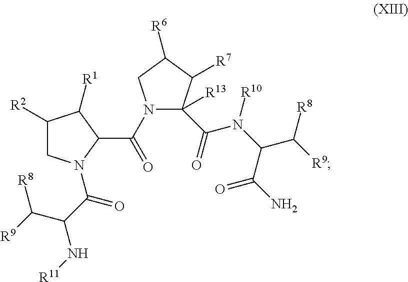

wherein:

R¹ and R² be independently selected from the group consisting of hydrogen; halogen; hydroxyl; substituted or unsubstituted C₁₋₆alkyl; substituted or unsubstituted C₁₋₆alkoxy; and substituted or unsubstituted aryl; or R¹ and R², together with the atoms to which they are attached, form a substituted or unsubstituted 4-6 membered heterocyclic or cycloalkyl ring;

R⁴, R⁵, and R¹² may be independently —C₁₋₆alkylene-phenyl, wherein C₁₋₆alkylene is optionally substituted by one or more substituents each independently selected from R^{f};

R⁶ and R⁷ may be independently selected from the group consisting of hydrogen; halogen; hydroxyl; substituted or unsubstituted C₁₋₆alkyl; substituted or unsubstituted C₁₋₆alkoxy; and substituted or unsubstituted aryl; or R⁶ and R⁷, together with the atoms to which they are attached, form a substituted or unsubstituted 4-6 membered heterocyclic or cycloalkyl ring;

R⁸ and R⁹ may be independently selected from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkylene-; naphthyl-C₁₋₆alkylene-; heteroaryl-C₁₋₆alkylene-; and heterocyclyl-C₁₋₆alkylene-; —OR^{x}; —NO₂; —N₃; —CN; —SCN; —SR^{x}; —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —N(R^{x})₂; —SOR^{x}; —S(O)₂R^{x}; —NR^{x}C(O)R^{x}; —NR^{x}C(O)N(R^{x})₂; —NR^{x}C(O)OR^{x}; —NR^{x}C(NR^{x})N(R^{x})₂; and —C(R^{x})₃; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl are each independently optionally substituted by one or more substituents each independently selected from R^{e}; wherein C₁₋₆alkyl and C₁₋₆alkylene are each independently optionally substituted by one or more substituents each independently selected from R^{f}; wherein C₃₋₆cycloalkyl is independently optionally substituted by one or more sub stituents each independently selected from R^{g};

R^{8a} and R^{9a} may be independently selected from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkylene-; naphthyl-C₁₋₆alkylene-; heteroaryl-C₁₋₆alkylene-; and heterocyclyl-C₁₋₆alkylene-; —OR^{x}; —ORY, —NO₂; —N₃; —CN; —SCN; —SR^{x}; —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —N(R^{x})₂; —SOR^{x}; —S(O)₂R^{x}; —NR^{x}C(O)R^{x}; —NR^{x}C(O)N(R^{x})₂; —NR^{x}C(O)OR^{x}; —NR^{x}C(NR^{x})N(R^{x})₂; and —C(R^{x})₃; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl are each independently optionally substituted by one or more substituents each independently selected from R^{e}; wherein C₁₋₆alkyl and C₁₋₆alkylene are each independently optionally substituted by one or more substituents each independently selected from R^{f}; wherein C₃₋₆cycloalkyl is independently optionally substituted by one or more substituents each independently selected from R^{g};

R¹⁰ and R¹¹ are independently selected from the group consisting of hydrogen; C₁₋₆alkyl; —C(O)—C₁₋₆alkylene; —C(O)—O—C₁₋₆alkylene; and —C(O)-phenyl; wherein C₁₋₆alkyl, C₁₋₆alkylene, and phenyl are optionally independently substituted by one or more substituents selected from R^{a};

R¹³ may be hydrogen or benzyl;

R^{b} may be selected, independently for each occurrence, from the group consisting of halogen; hydroxyl; —NO₂; —N₃; —CN; —SCN; C₁₋₆alkyl; C₂₋₆alkenyl; C₂₋₆alkynyl; C₃₋₆cycloalkyl; C₁₋₆alkoxy; C₃₋₆alkenyloxy; C₃₋₆alkynyloxy; C₃₋₆cycloalkoxy; C₁₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N—; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;

R^{a} and R^{a′} may be selected, independently for each occurrence, from the group consisting of hydrogen and C₁₋₆alkyl, or R^{a} and R^{a′} when taken together with the nitrogen to which they are attached form a 4-6 membered heterocyclic ring, wherein C₁₋₆alkyl is optionally substituted by one or more substituents each independently selected from the group consisting of halogen, oxo, and hydroxyl, and wherein the heterocyclic ring is optionally substituted by one or more substituents each independently selected from the group consisting of halogen, alkyl, oxo, or hydroxyl;

R^{c} may be selected, independently for each occurrence, from the group consisting of halogen; hydroxyl; —NO₂; —N₃; —CN; —SCN; oxo; C₁₋₆alkyl; C₂₋₆alkenyl; C₂₋₆alkynyl; C₃₋₆cycloalkyl; C₁₋₆alkoxy; C₃₋₆alkenyloxy; C₃₋₆alkynyloxy; C₃₋₆cycloalkoxy; C₁₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N—; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;

R^{d} may be selected, independently for each occurrence, from the group consisting of C₁₋₆alkyl, C₁₋₆alkylcarbonyl, and C₁₋₆alkylsulfonyl, wherein C₁₋₆alkyl is optionally substituted by one or more substituents each independently selected from halogen, hydroxyl, and R^{a}R^{a′}N—;

R^{e} may be selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;

R^{f} may be selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;

R^{g} may be selected, independently for each occurrence, from the group consisting of halogen, hydroxyl, —NO₂; —N₃; —CN; —SCN; C₁₋₆alkyl; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2; and

R^{x} may be hydrogen, halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkyl-; naphthyl-C₁₋₆alkyl-; heteroaryl-C₁₋₆alkyl-; and heterocyclyl-C₁₋₆alkyl-; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl, are each independently optionally substituted by one or more substituents each independently selected from R^{e}; wherein C₁₋₆alkyl is optionally substituted by one or more substituents each independently selected from R^{f}; wherein C₃₋₆cycloalkyl is independently optionally substituted by one or more substituents each independently selected from R^{g}; and

R^{y} may be selected, independently, from the group consisting of alkylsilyl, arylsilyl and heteroarylsilyl, wherein the heteroaryl of heteroarylsilyl may be a 5-6 membered ring having one, two, or three heteroatoms each independently selected from N, O, or S and may be optionally substituted with one or more substituents each independently selected from R^{b}; wherein the alkyl or aryl of the alkylsilyl or arylsilyl may optionally substituted by one or more sub stituents each independently selected from R^{f}.

In one embodiment, at least one of R^{8a} and R^{9a} is selected from —OR^{y}.

In some embodiments, R¹ and R² may be hydrogen. In certain embodiments, R⁶ and R⁷ may be hydrogen. In some instances, R¹⁰ and/or R¹¹ may be hydrogen.

In some embodiments, one of R⁸ or R⁹ may be hydrogen.

In some embodiments, R¹³ is hydrogen.

In certain embodiments, the compound of Formula IV may be

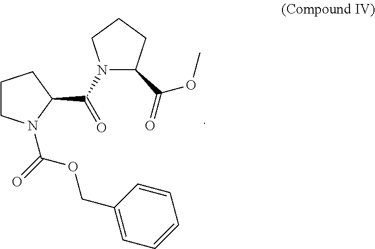

The compound of Formula V may be, for example,

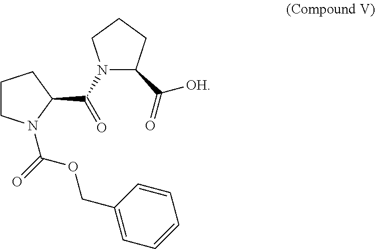

One non-limiting example of a compound of Formula VIII is

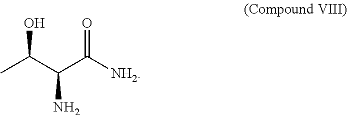

A compound of Formula IX may be exemplified by

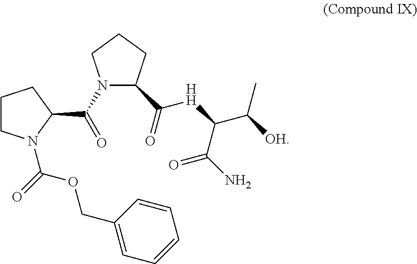

In some embodiments, a compound of Formula X may be

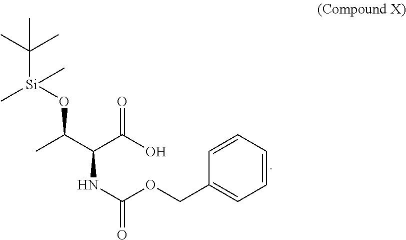 [SMILES: C[C@@H](O[Si](C)(C)C(C)(C)C)[C@H](NC(=O)OCc1ccccc1)C(=O)O]

In some cases, a compound of Formula XI may be

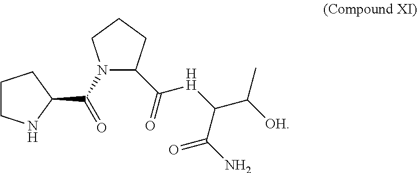

One non-limiting example of a compound of Formula XII is

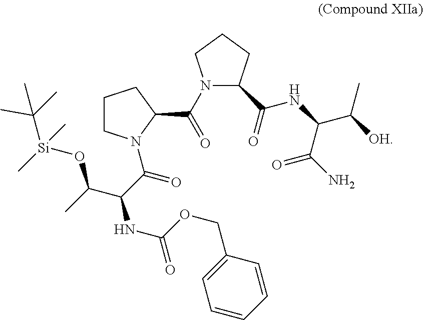 [SMILES: C[C@@H](O)[C@H](NC(=O)[C@@H]1CCCN1C(=O)[C@@H]1CCCN1C(=O)[C@@H](NC(=O)OCc1ccccc1)[C@@H](C)O[Si](C)(C)C(C)(C)C)C(N)=O]

In some embodiments, the compound of Formula XII may be produced by contacting a compound of Formula XIIa with a silyl ether cleaving reagent.

In some embodiments, the compound of Formula X may be produced by contacting a compound of Formula VI:

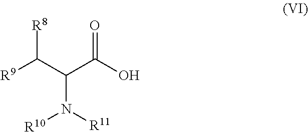

with an activated carbonyl compound. In certain embodiments, a base may be included in the reaction between the compound of the Formula VI and the activated carbonyl compound.

An activating agent may be any reagent capable of activating a carboxyl group for nucleophilic substitution. For example, in some embodiments, the activating agent may be used to convert the carboxyl group to an acyl halide, which may then undergo nucleophilic substitution. For instance, the reagent SOCl₂ may be used to convert the carboxyl group to an acyl chloride. In another embodiment, a carbodiimide may be used to activate a carboxyl group. For example, 1-ethyl-3-(3-dimethyllaminopropyl)carbodiimide (i.e., EDC), N,N′-dicyclohexylcarbodiimide (i.e., DCC), or N,N′-diisopropylcarbodiimide (i.e., DIC) may be used. In some embodiments, a carbodiimide-activated carboxyl group may be reacted to form an activated carbonyl group having more stability than a carbodiimide-activated carboxyl group. For example, the carbodiimide-activated carboxyl group may be reacted with N-hydroxysuccinimide or a suitable alternative thereof to form a less labile activated carbonyl group. In some embodiments, a chlorotriazine may be used to activate a carboxyl group. For example, 2-chloro-4,6-dimethoxy-1,3,5-triazine (i.e., CDMT), or 2,4,6-trichloro-1,3,5-triazine (TCT) may be used. In some embodiments, an alkyl chloroformate may be used to activate a carboxyl group. For example, methyl, ethyl or isobutyl chloroformates may be used.

An activated carbonyl compound may be reacted with a nucleophile to form, for example, an ester or amide. For example, in some embodiments, the activated carbonyl compound may be reacted with an alcohol (e.g., methanol, ethanol, or any other suitable alcohol) to form, for example, an ester or carbonate. In other embodiments, the activated carbonyl may be reacted with an amine to form, for example, an amide or carbamate. In one embodiment, the activated carbonyl compound may be a compound capable of forming a hydrogenation-labile carbonate or carbamate, e.g., benzyl chloroformate (i.e., Cbz-Cl).

In certain embodiments, reaction of an activated carbonyl compound with a nucleophile generates acid as a byproduct. For example, reaction of an acyl chloride with an alcohol or amine generates hydrochloric acid. In certain embodiments, it may be desirable to include a suitable acid scavenger in an acylation reaction. For example, a base such as a hydroxide salt (e.g., lithium hydroxide, sodium hydroxide, and the like), a carbonate (e.g., sodium carbonate, calcium carbonate, magnesium carbonate, and the like), or a bicarbonate (e.g., sodium bicarbonate) may be used.

A reagent capable of effecting hydrolysis may be any suitable reagent having this property. For example, the reagent may be a base such as a hydroxide salt (e.g., lithium hydroxide, sodium hydroxide, and the like).

A carbamate-cleaving reagent may be any suitable reagent capable of liberating an amine from a carbamate. The reagent may be chosen, for example, based on the identity of the carbamate. For instance, a base (e.g., a hydroxide salt) may be used to hydrolyze a carbamate. In embodiments where the carbamate comprises an alkyl-aryl ester (e.g., a benzyl ester), the carbamate-cleaving reagent may be a catalytic hydrogenation reagent (e.g., palladium on carbon (Pd/C)).

A silyl ether cleaving reagent may be any suitable reagent capable of liberating an alcohol from a silyl ether. The reagent may be chosen, for example, based on the identity of the silyl ether. For instance, an acid (e.g., HCl) or a fluoride (e.g., tetrabutyl ammonium fluoride) may be used to cleave a silyl ether.

Each of the steps of the processes contemplated herein may be performed at any suitable temperature or gradient of temperatures. For example, a reaction may be carried out at a temperature of between about −20° C. to about 150° C., in some embodiments about 0° C. to about 100° C., in some embodiments between 15° C. and about 30° C., in some embodiments between about −10° C. to about 30° C., in some embodiments between about −20° C. to about 0° C., in some embodiments between about 0° C. to about 30° C., in some embodiments between about 0° C. to about 5° C., and in some embodiments between about 20° C. to about 30° C.

In certain embodiments, a lyophilization step may be included in the process. For example, the compound of Formula XIII may be lyophilized. Lyophilizing may be carried out at any suitable temperature or gradient of temperatures. For example, the lyophilization may be carried at a temperature of between about −50° C. to about 25° C. In some instances, the temperature may be increased from a first temperature of about −60° C. to about −40° C. to a second temperature of about 15° C. to about 30° C. The temperature gradient may occur over any suitable period of time. For example, in some embodiments, the period of time may be about 4 to about 200 hours, or may be about 4 to about 48 hours, in some embodiments about 12 to about 36 hours, or in some embodiments about 20 to about 30 hours.

### Definitions

In some embodiments, the compounds, as described herein, may be substituted with any number of substituents or functional moieties. In general, the term “substituted” whether preceded by the term “optionally” or not, and substituents contained in formulas, refer to the replacement of hydrogen radicals in a given structure with the radical of a specified substituent.

In some instances, when more than one position in any given structure may be substituted with more than one substituent selected from a specified group, the substituent may be either the same or different at every position.

As used herein, the term “substituted” is contemplated to include all permissible sub stituents of organic compounds. In a broad aspect, the permissible substituents include acyclic and cyclic, branched and unbranched, carbocyclic and heterocyclic, aromatic and non-aromatic substituents of organic compounds. In some embodiments, heteroatoms such as nitrogen may have hydrogen substituents and/or any permissible substituents of organic compounds described herein which satisfy the valencies of the heteroatoms. Non-limiting examples of substituents include acyl; aliphatic; heteroaliphatic; aryl; heteroaryl; arylalkyl; heteroarylalkyl; alkoxy; cycloalkoxy; heterocyclylalkoxy; heterocyclyloxy; heterocyclyloxyalkyl; alkenyloxy; alkynyloxy; aryloxy; heteroalkoxy; heteroaryloxy; alkylthio; arylthio; heteroarylthio; oxo; —F; —Cl; —Br; —I; —OH; —NO₂; —N₃; —CN; —SCN; —SR^{x}; —CF₃; —CH₂CF₃; —CHCl₂; —CH₂OH; —CH₂CH₂OH; —CH₂NH₂; —CH₂SO₂CH₃; —OR^{x}, —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —N(R^{x})₂; —SOR^{x}; —S(O)₂R^{x}; —NR^{x}C(O)R^{x}; —NR^{x}C(O)N(R^{x})₂; —NR^{x}C(O)OR^{x}; —NR^{x}C(NR^{x})N(R^{x})₂; and —C(R^{x})₃; wherein each occurrence of R^{x} independently includes, but is not limited to, hydrogen, halogen, acyl, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Furthermore, the compounds described herein are not intended to be limited in any manner by the permissible substituents of organic compounds. In some embodiments, combinations of substituents and variables described herein may be preferably those that result in the formation of stable compounds. The term “stable,” as used herein, refers to compounds which possess stability sufficient to allow manufacture and which maintain the integrity of the compound for a sufficient period of time to be detected and preferably for a sufficient period of time to be useful for the purposes detailed herein.

The term “acyl,” as used herein, refers to a moiety that includes a carbonyl group. In some embodiments, an acyl group may have a general formula selected from —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —NR^{x}C(O)R^{x}; —NR^{x}C(O)N(R^{x})₂; and —NR^{x}C(O)OR^{x}; wherein each occurrence of R^{x} independently includes, but is not limited to, hydrogen, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “aliphatic,” as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties.

The term “heteroaliphatic,” as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles (e.g., morpholino, pyrrolidinyl, etc.), which may be optionally substituted with one or more functional groups or may be unsubstituted.

The terms “aryl” and “heteroaryl,” as used herein, refer to mono- or polycyclic unsaturated moieties having preferably 3-14 carbon atoms, each of which may be substituted or unsubstituted. In certain embodiments, “aryl” refers to a mono- or bicyclic carbocyclic ring system having one or two aromatic rings including, but not limited to, phenyl, naphthyl, tetrahydronaphthyl, indanyl, indenyl, and the like. In certain embodiments, “heteroaryl” refers to a mono- or bicyclic heterocyclic ring system having one or two aromatic rings in which one, two, or three ring atoms are heteroatoms independently selected from the group consisting of S, O, and N and the remaining ring atoms are carbon. Non-limiting examples of heteroaryl groups include pyridyl, pyrazinyl, pyrimidinyl, pyrrolyl, pyrazolyl, imidazolyl, thiazolyl, oxazolyl, isooxazolyl, thiadiazolyl,oxadiazolyl, thiophenyl, furanyl, quinolinyl, isoquinolinyl, and the like.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂alkenyl, C₂-C₁₀alkenyl, and C₂-C₆alkenyl, respectively. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, etc.

The term “alkenyloxy” used herein refers to a straight or branched alkenyl group attached to an oxygen (alkenyl-O). Exemplary alkenoxy groups include, but are not limited to, groups with an alkenyl group of 3-6 carbon atoms referred to herein as C₃₋₆alkenyloxy. Exemplary “alkenyloxy” groups include, but are not limited to allyloxy, butenyloxy, etc.

The term “alkoxy” as used herein refers to an alkyl group attached to an oxygen (—O-alkyl). Exemplary alkoxy groups include, but are not limited to, groups with an alkyl group of 1-12, 1-8, or 1-6 carbon atoms, referred to herein as C₁-C₁₂alkoxy, C₁-C₈alkoxy, and C₁-C₆alkoxy, respectively. Exemplary alkoxy groups include, but are not limited to methoxy, ethoxy, etc. Similarly, exemplary “alkenoxy” groups include, but are not limited to vinyloxy, allyloxy, butenoxy, etc.

The term “alkoxycarbonyl” as used herein refers to a straight or branched alkyl group attached to oxygen, attached to a carbonyl group (alkyl-O—C(O)—). Exemplary alkoxycarbonyl groups include, but are not limited to, alkoxycarbonyl groups of 1-6 carbon atoms, referred to herein as C₁₋₆alkoxycarbonyl. Exemplary alkoxycarbonyl groups include, but are not limited to, methoxycarbonyl, ethoxycarbonyl, t-butoxycarbonyl, etc.

The term “alkynyloxy” used herein refers to a straight or branched alkynyl group attached to an oxygen (alkynyl-O)). Exemplary alkynyloxy groups include, but are not limited to, propynyloxy.

The term “alkyl” as used herein refers to a saturated straight or branched hydrocarbon, for example, such as a straight or branched group of 1-6, 1-4, or 1-3 carbon atom, referred to herein as C₁-C₆alkyl, C₁-C₄alkyl, and C₁-C₃alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc. For example, alkyl may refer to a C₁₋₆alkyl, optionally substituted by one, two, or three substituents selected from the group consisting of: halo, nitro, hydroxyl, —NH₂, —NH-alkyl, or alkoxy (e.g. —OCH₃).

The term “alkylcarbonyl” as used herein refers to a straight or branched alkyl group attached to a carbonyl group (alkyl-C(O)—). Exemplary alkylcarbonyl groups include, but are not limited to, alkylcarbonyl groups of 1-6 atoms, referred to herein as C₁-C₆alkylcarbonyl groups. Exemplary alkylcarbonyl groups include, but are not limited to, acetyl, propanoyl, isopropanoyl, butanoyl, etc.

The term “alkynyl” as used herein refers to an unsaturated straight or branched hydrocarbon having at least one carbon-carbon triple bond, such as a straight or branched group of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

Alkyl, alkenyl and alkynyl groups can optionally be substituted, if not indicated otherwise, with one or more groups selected from alkoxy, alkyl, cycloalkyl, amino, halogen, and —C(O)alkyl. In certain embodiments, the alkyl, alkenyl, and alkynyl groups are not substituted, i.e., they are unsubstituted.

The term “amide” or “amido” as used herein refers to a radical of the form —R^{a}C(O)N(R^{b})—, —R^{a}C(O)N(R^{b})R^{c}—, or —C(O)NR^{b}R^{c}, wherein R^{a}, R^{b}, and R^{c} are each independently selected from alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, and nitro. The amide can be attached to another group through the carbon, the nitrogen, R^{b}, R^{c}, or R^{a}. The amide also may be cyclic, for example R^{b} and R^{c}, R^{a} and R^{b}, or R^{a} and R^{c} may be joined to form a 3- to 12-membered ring, such as a 3- to 10-membered ring or a 5- to 6-membered ring. The term “carboxamido” refers to the structure —C(O)NR^{b}R^{c}.

The term “amine” or “amino” as used herein refers to a radical of the form —NR^{d}R^{e}, where R^{d} and R^{e} are independently selected from hydrogen, alkyl, alkenyl, alkynyl, aryl, arylalkyl, cycloalkyl, haloalkyl, heteroaryl, and heterocyclyl. The amino also may be cyclic, for example, R^{d} and R^{e} are joined together with the N to form a 3- to 12-membered ring, e.g., morpholino or piperidinyl. The term amino also includes the corresponding quaternary ammonium salt of any amino group, e.g., —[N(R^{d})(R^{e})(R^{f})]+. Exemplary amino groups include aminoalkyl groups, wherein at least one of R^{d}, R^{e}, or R^{f} is an alkyl group. In certain embodiment, R^{d} and R^{e} are hydrogen or alkyl.

The term “cycloalkoxy” as used herein refers to a cycloalkyl group attached to an oxygen (cycloalkyl-O—).

The term “cycloalkyl” as used herein refers to a monocyclic saturated or partially unsaturated hydrocarbon group of for example 3-6, or 4-6 carbons, referred to herein, e.g., as C₃₋₆cycloalkyl or C₄₋₆cycloalkyl and derived from a cycloalkane. Exemplary cycloalkyl groups include, but are not limited to, cyclohexyl, cyclohexenyl, cyclopentyl, cyclobutyl or, cyclopropyl.

The terms “halo” or “halogen” or “Hal” as used herein refer to F, Cl, Br, or I. The term “haloalkyl” as used herein refers to an alkyl group substituted with one or more halogen atoms.

The terms “heterocyclyl” or “heterocyclic group” are art-recognized and refer to saturated or partially unsaturated 3- to 10-membered ring structures, alternatively 3- to 7-membered rings, whose ring structures include one to four heteroatoms, such as nitrogen, oxygen, and sulfur. Heterocycles may also be mono-, bi-, or other multi-cyclic ring systems. A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isoxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. The heterocyclic ring may be substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl. In certain embodiments, the heterocyclic group is not substituted, i.e., the heterocyclic group is unsubstituted.

The term “heteroaryloxy” refers to a heteroaryl-O— group.

The term “heterocycloalkyl” is art-recognized and refers to a saturated heterocyclyl group as defined above. The term “heterocyclylalkoxy” as used herein refers to a heterocyclyl attached to an alkoxy group. The term “heterocyclyloxyalkyl” refers to a heterocyclyl attached to an oxygen (—O—), which is attached to an alkyl group.

The term “heterocyclylalkoxy” as used herein refers to a heterocyclyl-alkyl-O—group.

The term “heterocyclyloxy” refers to a heterocyclyl-O— group.

The term “heterocyclyloxyalkyl” refers to a heterocyclyl-O-alkyl-group.

The terms “hydroxy” and “hydroxyl” as used herein refers to the radical —OH.

The term “oxo” as used herein refers to the radical ═O.

The term “alkylsilyl” refers to one or more alkyl, as defined above, attached to —Si. For example, alkylsilyl may include —SiH₂R, —SiHRR′, or —SiRR′R″, in which one of R, R′ and R″ are alkyl groups, which can be the same or different . The groups, —SiH₂CH₃, —SiH(CH₃)₂, —Si(CH₃)₃ and —Si(CH₃)₂C(CH₃)₃, are non-limiting examples of alkylsilyl groups. So long as R is an alkyl group, R′ and R″ may constitute a non-alkyl group, such as aryl or hereteroaryl. Further, at least one of R, R′ and R″ may be substituted. For further example, alkylsilyl may refer to a C₁₋₆ alkyl attached to Si, where the C₁₋₆ alkyl is substituted by one, two, or three substituents selected from the group consisting of: halo, nitro, hydroxyl, —NH₂, —NH-alkyl, or alkoxy (e.g. —OCH₃).

The term “arylsilyl” refers to one or more aryl, as defined above, attached to —Si. For example, arylsilyl may include —SiH₂(phenol), —SiH(phenol)₂, or —Si(phenol)₃, where each phenol may be the same or different. Further, arylsilyl may include —Si(aryl)R′R″, where, R′ and R″ may constitute a non-aryl group, such as alkyl or hereteroaryl.

The term “heteroarylsilyl” refers to one or more herteroaryl, as defined above, attached to —Si. For example, heteroarylsilyl may include —SiH₂(heteroaryl), —SiH(heteroaryl)₂, or —Si(heteroaryl)₃, where each heteroaryl may be the same or different. Further, heteroarylsilyl may include —Si(heteroaryl)R′R″, where, R′ and R″ may constitute a non-heteroaryl group, such as alkyl or aryl.

“Pharmaceutically or pharmacologically acceptable” include molecular entities and compositions that do not produce an adverse, allergic or other untoward reaction when administered to an animal, or a human, as appropriate. “For human administration, preparations should meet sterility, pyrogenicity, general safety and purity standards as required by FDA Office of Biologics standards.

As used in the present disclosure, the term “partial NMDA receptor agonist” is defined as a compound that is capable of binding to a glycine binding site of an NMDA receptor; at low concentrations a NMDA receptor agonist acts substantially as agonist and at high concentrations it acts substantially as an antagonist. These concentrations are experimentally determined for each partial agonist.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

For simplicity, chemical moieties that are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms alkoxy“, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The term “compound” as used herein all include pharmaceutically acceptable salts, co-crystals, solvates, hydrates, polymorphs, enantiomers, diastereoisomers, racemates and the like of the compounds.

The term “pharmaceutically acceptable salt(s)” as used herein refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts. The term “salts” include “pharmaceutically acceptable salts.”

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well known asymmetric synthetic methods.

Geometric isomers can also exist in the compounds of the present invention. The symbol [symbol] denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “F” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The compounds disclosed herein can exist in solvated as well as unsolvated forms with pharmaceutically acceptable solvents such as water, ethanol, and the like, and it is intended that the invention embrace both solvated and unsolvated forms. In one embodiment, the compound is amorphous. In one embodiment, the compound is a polymorph. In another embodiment, the compound is in a crystalline form.

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in the e.g., Examples herein by substituting an isotopically labeled reagent for a non-isotopically labeled reagent.

As used in the present disclosure, “NMDA” is defined as N-methyl-D-aspartate.

### EXAMPLES

The following examples are provided for illustrative purposes only, and are not intended to limit the scope of the disclosure.

### Example 1

### Preparation of Methyl L-Prolinate Hydrochloride (Compound II)

To a solution of L-Proline (Compound I, 5 kg, 43.4 moles) in methanol (25 L) was charged thionyl chloride (7.75 kg, 65.1 moles) at 0-5° C. The reaction mixture was stirred overnight at room temperature and concentrated under reduced pressure below 50° C. Toluene (5 L) was added and the mixture was distilled and degasified for 2-4 hours under reduced pressure at 50° C.

¹H-NMR: (500 MHz, DMSO-d₆): δ 9.09 (s, 1H), 4.35-4.24 (m, 1H), 3.75 (s, 3H), 3.21-3.16 (m, 2H), 2.28-2.21 (m, 2H), 2.01-1.88 (m, 2H).

### Example 2

### Preparation of ((Benzyloxy)carbonyl)-L-Proline (Compound III)

A solution of NaOH (6.94 kg, 172.5 moles) in water (35 L) was prepared and added slowly to the reaction mixture obtained from Example 1. The resulting biphasic solution was cooled to 0-5° C. and reacted with a 50% solution of benzyl chloroformate (11.37 L, 47.7 moles) in toluene for 3-4 hours. MTBE (20 L) was added to the reaction mass at 20-30° C. The reaction mixture was stirred and allowed to settle. After separation of both layers, the aqueous layer was acidified to pH 1-2 and extracted with ethyl acetate. The organic layer was washed with brine solution, dried over sodium sulfate and concentrated under reduced pressure to obtain compound III (10.2 kg, 94% from Compound I).

¹1-1-NMR (500 MHz, DMSO-d₆): δ 12.85-12.58 (br s, 1H), 7.38-7.28 (m, 5H), 5.12-4.93 (m, 2H), 4.28-4.15 (dd, 1H), 3.45-3.30 (m, 2H), 2.32-2.09 (m, 1H), 1.92-1.68 (m, 3H)

### Example 3

### Preparation of (S)-Benzyl 2-((S)-2-(methoxycarbonyl) pyrrolidine-1-carbonyl)-pyrrolidine-1-carboxylate (Compound IV)

A solution of Compound I (L-Proline, 60 g, 0.52 moles) in methanol (810 mL) was cooled to 0-5° C. and treated with thionyl chloride (100 mL, 1.37 moles). The reaction mixture was stirred for 6-8 hours at 20-35° C. to obtain Compound II. Reaction mixture was distilled to 1-2 volumes under reduced pressure at below 40° C. A series of toluene additions and distillations were performed under reduced pressure at below 50° C. to 1-2 volumes. Dichloromethane (1.0 L) was then added at 25-35° C.

In another reactor, the Compound III (100 g, 0.4 moles) was dissolved in Dichloromethane (500 mL) at below 20° C. and the resulting solution was cooled to 0-5° C. N-Methyl morpholine (NMIM, 57 mL, 0.52 moles) was added slowly to it at 0-5° C. 2-Chloro-4,6-dimethoxy-1,3,5-triazine (CDMT, 74 g, 0.42 moles) was then added slowly to this and the reaction mixture was stirred overnight at room temperature. N-Methyl morpholine (NMM, 88 mL, 0.8 moles) was added slowly to the reaction mixture at 0-5° C. Above prepared L-Proline methyl ester hydrochloride (Compound II) solution in DCM was gradually added to the reaction mixture containing the active ester of Compound III at −10 to 5° C. The temperature of the coupling reaction was increased to room temperature and the mixture was stirred for 3-4 h. The reaction mass was filtered and washed with DCM (200 mL). (Note: By product HDMT was filtered). Combined organic layer was washed with aq. sodium bicarbonate, water and 6N HCl. The organic layer was distilled under reduced pressure at below 45° C. to 1-2 volumes. THF (500 mL) was added and the mixture was distilled under reduced pressure to 1-2 volumes at below 45° C. A second lot of THF (500 mL) was added and the mixture was distilled under reduced pressure to 1-2 volumes at below 45° C. to obtain Compound IV (8 kg, 85%)

¹H-NMR (400 MHz, DMSO-d₆): δ 7.39-7.25 (m, 5H), 5.08-4.88 (m, 2H), 4.53-4.49 (m, 1H), 4.24 (dq, 1H), 3.70-3.64 (m, 1H), 3.57 (s, 3H), 3.55-3.38 (m, 3H), 2.27-1.66 (m, 8H).

### Example 4

### Preparation of (S)-1-((S)-1-(Benzyloxycarbonyl) pyrrolidine-2-carbonyl) pyrrolidine-2-carboxylic Acid (Compound V)

To a mixture of THF (500 mL) and water (500 mL) was charged the solution of Compound IV in THF, obtained from Example 3. Lithium Hydroxide (20.2 g, 0.48 moles) was added to the reaction mixture and stirred at room temperature overnight. Reaction mixture was washed with MTBE and pH of aqueous layer was adjusted to 1.0-2.0 with concentrated HCl. The resulting slurry was stirred for 1-2 hours at 0-5° C. and filtered. The filter cake was washed with water and MTBE and then dried to obtain Compound V (122 g, 86%).

¹⁻H-NMR (400 MHz, DMSO-d₆): δ 7.39-7.25 (m, 5H), 5.08-4.88 (m, 2H), 4.53-4.49 (m, 1H), 4.24 (dq, 1H), 3.70-3.64 (m, 1H), 3.57 (s, 3H), 3.55-3.38 (m, 3H), 2.27-1.66 (m, 8H).

### Example 5

### Preparation of Methyl L-threoninate (Compound VII)

To a cooled solution of Compound VI (L-threonine, 5 kg, 16.7 moles) in methanol (25 L) was added thionyl chloride (7.45 kg, 25 moles) and the reaction mixture was stirred overnight at 20-25° C. It was then concentrated to a residue and dissolved in methanol (5 L). The methanolic solution was again concentrated to a residue under reduced pressure at below 50° C. and degasified to obtain Compound VII.

### Example 6

### Preparation of (2S, 3R)-2-Amino-3-hydroxybutanamide (Compound VIII)

Isopropanol (35 L) was added to the reaction mixture obtained from Example 5. The resulting solution was charged into an autoclave and ammonia gas pressure (4.5-5 Kg) was applied to the reaction mixture at room temperature overnight. It was then filtered, washed with isopropanol (10 L) and filtrate was distilled under reduced pressure to a residue. MTBE (15+5 L) was added slowly and the resulting slurry was stirred and filtered. The filtered cake was dried to obtain Compound VIII (3 kg, 70% from Compound VI).

¹H-NMR: (500 MHz, DMSO-d₆): δ 7.37 (brs, 1H), 7.02 (brs, 1H), 3.75 (q, 1H), 2.95 (d, 1H), 1.05 (d, 3H).

### Example 7

### Preparation of (S)-Benzyl 2-((S)-2-((2S, 3R)-1-amino-3-hydroxy-1-oxobutan-2-ylcarbamoyl) pyrrolidine-1-carbonyl) pyrrolidine-1-carboxylate (Compound IX)

To a cooled solution of Compound V (10 g, 28.87 mmol) in dichloromethane (100 mL) were charged N-Methyl morpholine (3.21 g, 31.76 mmol) and Isobutylchloroformate (4.33 g, 31.76 mmol). After stirring the mixture for 1-2 h, a second lot of N-Methyl morpholine (3.21 g, 31.76 mmol) was added. Compound VIII (4.43 g, 37.52 mmol) was then added and the reaction mixture was left with stirring overnight at room temperature. Brine (30 mL) was charged into the reaction mass, separated the layers and the aqueous layer was extracted with dichloromethane (50 mL). Organic layers were combined and washed with brine solution. The organic layer was concentrated under reduced pressure. The reaction mixture was charged with isopropanol (100 mL) and concentrated to 30-40 mL volume under vacuum. Isopropanol (100 mL) was added and concentrated to 30-40 mL volume under vacuum. The mixture was cooled and the product (Compound IX) was filtered, washed with pre-cooled isopropanol and dried (10.3 g, 78%).

¹H-NMR: (500 MHz, DMSO-d₆): δ 7.40-7.25 (m, 5H), 7.15-7.02 (d, 2H), 5.12-4.82 (m, 3H), 4.60-4.52 (m, 1H), 4.42-4.30 (dd, 1H), 4.08-3.96 (m, 2H), 3.70-3.33 (m, 4H), 2.30-1.60 (m, 8H), 1.05 (d, 3H).

### Example 7a

### Preparation of (S)-Benzyl 2-((S)-2-((2S, 3R)-1-amino-3-hydroxy-1-oxobutan-2-ylcarbamoyl) pyrrolidine-1-carbonyl) pyrrolidine-1-carboxylate (Compound IX)

To a slurry of Compound VIII.HCl (27.8 g, 0.18 mol) in dichloromethane (900 mL) was added triethylamine (42.5 g, 0.42 mol). The resulting mixture was stirred at room temperature for 1-2 hours. Compound V (50 g, 0.14 mol) was then introduced in one portion. To this, a solution of Isobutylchloroformate (20.5 g, 0.15 mol) in dichloromethane (100 mL) was charged at room temperature over 6 hours. The reaction mixture was then left with stirring for 3-4 hours at room temperature. Brine (2×250 mL) was charged into the reaction mass, layers were separated and the aqueous layer was extracted with dichloromethane (100 mL). The combined organic layer was concentrated under reduced pressure. The reaction mixture was charged with isopropanol (500 mL) and concentrated to 200-250 mL volume under vacuum. Isopropanol (500 mL) was added and concentrated to 200-250 mL volume under vacuum. The mixture was cooled and the product (Compound IX) was filtered, washed with pre-cooled isopropanol and dried (55.4 g, 86%).

### Example 8

### Preparation of ((Benzyloxy)carbonyl)-L-threonine (Compound Xa)

To a mixture of sodium bicarbonate (28.18 kg, 336 moles) and water (50 L) was charged Compound VI (L-threonine, 10 kg, 84 moles) Benzyl chloroformate (31.4 L, 92 moles) was then added at 0-5° C. and the reaction mixture was stirred overnight. After adding MTBE (30 L), reaction mixture was stirred, layers were separated. The aqueous layer was washed with toluene (20 L) and MTBE (20 L) and then the pH of aqueous layer was adjusted to 1.0-2.0 with concentrated HCl. The reaction mass was stirred for 15 min, then ethyl acetate (30 L) was added. The organic layers were separated and the aqueous layer was extracted with ethyl acetate (20 L). The organic layers were combined and washed with brine solution. Ethyl acetate (100 L) was added to the organic layer. Dicyclohexylamine (30.42 kg, 168 moles) was added and the reaction mixture was stirred at room temperature for 4-5 h. The resulting slurry was cooled, filtered and the filter cake was washed with ethyl acetate (100 L). It was slurried in water (250 L), and the pH was adjusted to 1.0-2.0 with 2N sulphuric acid. The reaction mixture was stirred while ethyl acetate (100 L) was added. The layers were separated and the aqueous layer was extracted with ethyl acetate (100 L). The combined organic layer was dried with sodium sulphate and filtered. The organic layer was concentrated to a residue under vacuum to obtain Compound Xa (9.6 kg, 45%).

¹⁻H-NMR: (500 MHz, DMSO-d₆): δ 12.75-12.55 (brs, 1H), 7.45-7.35 (m, 5H), 6.95 (d, 1H), 5.15 (s, 2H), 4.82-4.60 (brs, 1H), 4.15 (q, 1H), 3.97 (d, 1H), 1.15 (d, 3H).

### Example 9

### Preparation of (2S, 3R)-2-(Benzyloxycarbonylamino)-3-tertiarybutyl dimethyl silyloxy-butanoic Acid (Compound X)

A mixture of Compound Xa (25.3 g, 100 mmol), imidazole (14.9 g, 220 moles) and TBDMSC1 (16.9 g, 113 mmol) in DMF (21 mL) was heated at 40-50° C. overnight. It was then added slowly with stirring to cold water (375 mL) and the resulting slurry was filtered and washed with cold water (75 ml) and heptane (75 ml). It was then dried under vacuum to obtain Compound X (18 g, 50%).

¹H-NMR: (500 MHz, DMSO-d₆): δ 12.75 (s, 1H), 7.66-7.20 (m, 5H), 6.72 (d, 1H), 5.12 (s, 2H), 4.30 (q, 1H), 4.04 (d, 1H), 1.13 (d, 3H), 0.82 (s, 9H), 0.03 (m, 6H).

### Example 10

### Preparation of (S)—N-((2S, 3R)-1-amino-3-hydroxy-1-oxobutan-2-yl)-1-((S)-pyrrolidine-2-carbonyl) pyrrolidine-2-carboxamide (Compound XI)

10% Palladium on Carbon (w/w, 50% wet; 5.75 g; 0.23 times, w/w) was charged into the pressure reactor at ambient temperature under nitrogen atmosphere. Compound IX (25 g, 56 mmol), ethanol (225 mL) and conc. HCl (5.6 mL, 67.2 mmol) were added to the reactor. Hydrogen pressure was maintained at 45-60 psi at ambient temperature for 5-6 h. Hyflow bed was prepared with ethanol (25 mL). The reaction mass was filtered under nitrogen atmosphere and filter bed was washed with ethanol (25 mL) to obtain Compound XI.

### Example 11

### Preparation of Benzyl (2S, 3R)-1-((S)-2-((S)-2-((2S, 3R)-1-amino-3-tertiary butyl dimethyl silyloxy-1-oxobutan-2-ylcarbamoyl) pyrrolidine-1-carbonyl) pyrrolidin-1-yl)-3-hydroxy-1-oxobutan-2-ylcarbamate (Compound XIIa)

An ethanolic solution of Compound XI (56 mmoL), obtained from Example 10, was charged into the reactor under stirring and reaction mixture was cooled to 0-5° C. EDC.HCl (13.9 g, 72.8 mmol) and HOBT (9.46 g, 61.6 mmol) were charged and reaction mixture was cooled to −5 to 0° C. N-Methyl morpholine (28.3 g, 280 mmol) was then added drop wise to the above reaction mixture. Compound X (22.64 g, 61.6 mmol) was charged into the reactor under stirring and reaction mixture was left with stirring overnight at room temperature. After completion of the coupling reaction, it was quenched with water (4 volumes) and distilled under vacuum to 3 volumes. Water (7 volumes) and dichloromethane (10 volumes) were charged and both the layers were separated. The organic layer containing coupled product was washed successively with 20% aqueous citric acid solution (10 volumes), water (10 volumes), saturated sodium bicarbonate solution (2×10 volumes) and water (10 volumes). It was then concentrated under vacuum at below 50° C. to 3 volumes. From the above reaction, a solution of Compound XIIa in methylene chloride was obtained.

¹H-NMR: (500 MHz, DMSO-d₆): δ 7.45-7.20 (m, 6H), 7.18-7.02 (d, 2H), 5.18-4.80 (m, 3H), 4.63-4.41 (m, 1H), 4.42-4.15 (m, 2H), 4.02 (s, 2H), 3.90-3.82 (m, 1H), 3.80-3.45 (m, 3H), 2.30-1.72 (m, 6H), 1.22-1.06 (d, 3H), 1.05-0.95 (d, 3H), 1.90-1.63 (m, 9H), 0.02 (m, 6H).

### Example 12

### Preparation of Benzyl (2S, 3R)-1-((S)-2-((S)-2-((2S, 3R)-1-amino-3-hydroxy-1-oxobutan-2-ylcarbamoyl) pyrrolidine-1-carbonyl) pyrrolidin-1-yl)-3-hydroxy-1-oxobutan-2-ylcarbamate (Compound XII)

A solution of Compound XIIa (56 mmoL) in methylene chloride, obtained from Example 11, was charged into a reactor. It was diluted with THF (125 mL) and the mixture was distilled under vacuum to 3 volumes (75 ml). A second lot of THF (125 mL) was added and the mixture was distilled under vacuum to 5 volumes (125 mL). It was cooled to 0-5° C. and then water (125 ml) and conc. HCl (9.25 ml) were added. After completion of the reaction (12-16 h), the pH of the mixture was adjusted to 5-6 using aq. sodium bicarbonate solution. Methyl t-butyl ether (250 mL) was charged into the reaction mass. Separated the layers and the aqueous layer was washed with methyl t-butyl ether (250 mL). Brine (50 mL) was charged and the product was extracted with dichloromethane (3×75 mL). Organic layers were combined and concentrated to 3 volumes (75 mL) under reduced pressure. The reaction mixture was cooled and charged dropwise to a stirred solution of methyl t-butyl ether (275 mL) to obtain a slurry of the product which was cooled, filtered, and the filter cake was washed with cold methyl t-butyl ether (50 mL) and dried under vacuum to obtain Compound XII (25 g, 85% from Compound XI).

¹H-NMR: (400 MHz, DMSO-d₆): δ 7.38-7.29 (m, 5H), 7.19-7.17 (d, 1H), 7.08-7.04 (d, 1H), 5.03-4.85 (m, 3H), 4.61-4.58 (m, 1H), 4.40-4.37 (m, 1H), 4.16-4.12 (t, 1H), 4.09-3.99 (m, 2H), 3.84-3.58 (m, 4H), 2.32-1.72 (m, 8H), 1.11 (d, 3H), 1.00 (d, 3H).

### Example 13

### Preparation of Benzyl (S)—N-((2S,3R)-1-amino-3-hydroxy-1-oxobutan-2-yl)-1-((S)-1-((2R,3R)-2-amino-3-hydroxybutanoyl) pyrrolidine-2 carbonyl) pyrrolidine-2-carboxamide (Rapastinel, Compound XIII)

10% Palladium Carbon (50% wet; 0.31 kg, 0.11 times, w/w) was charged into the pressure reactor at ambient temperature under nitrogen atmosphere. Compound XII (2.8 kg, 5.11 moles) was dissolved in ethanol (22 L) and added to the reactor. Hydrogen pressure was maintained at 3-4 kg/cm² at ambient temperature over a period of 4-8 hrs. Prepared the hyflow bed (0.28 kg) with ethanol (6 L) and the reaction mass was filtered through hyflow bed under nitrogen atmosphere, and the filtrate was collected into a clean HDPE container. The filter bed was washed with ethanol (15 L) and the filtrate was concentrated under reduced pressure to 2 volumes. The ethanolic solution was charged with Dichloromethane (9 L) and stirred till dissolution. MTBE (60L) was charged to another reactor and stirred. The Above EtOH/DCM solution was added to stirred MTBE slowly. During the addition, the product, rapastinel was precipitated. The resulting suspension was filtered, and the filter cake was washed with MTBE (2×15 L) and dried under vacuum to obtain rapastinel (2.1 kg, 95%) (Compound XIII).

¹H-NMR: (400 MHz, DMSO-d₆): δ 7.37 (d, 1H), 7.06 (d, 2H), 4.96-4.86 (m, 1H), 4.58 (dd, 1H), 4.45-4.34 (m, 1H), 4.07-3.99 (m, 2H), 3.70-3.65 (m, 2H), 3.61-3.54 (m, 2H),3.48-3.43 (m, 1H), 3.28-3.22 (m, 1H), 2.32-1.68 (m, 8H), 1.07 (d, 3H), 1.00 (d, 3H).

### Example 14

### Lyophilization of Rapastinel

Rapastinel pure product (3.0 kg, 7.3 moles)) obtained from Stage D was dissolved in water (10 L, 30% w/w)) and stirred for 30 minutes at 20-25° C. It was then washed with MTBE (2×30 L) to remove trace amounts of toluene, ethanol and dichloromethane. The aqueous rapastinel solution is vacuum distilled to remove trace amounts of MTBE. The product rich aqueous solution is polish-filtered. The resulting rapastinel solution is charged into lyophilization trays, freeze-dried until water content is NMT 5.0% w/w, and the final lyophilized rapastinel drug substance (2.8 kg, 95%) is packaged and stored.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications, websites, and other references cited herein are hereby expressly incorporated herein in their entireties by reference.

What is claimed is:

1. A process for synthesizing a dipyrrolidine peptide compound or a pharmaceutically acceptable salt, stereoisomer, metabolite, or hydrate thereof, comprising the steps:
a) contacting a compound of Formula III, or a salt thereof: 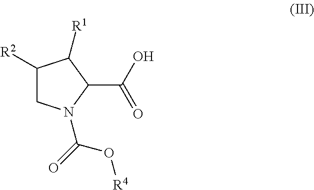
with an activating reagent and a compound of Formula II, or a salt thereof: 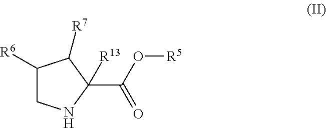
to produce a compound of Formula IV, or a salt thereof: 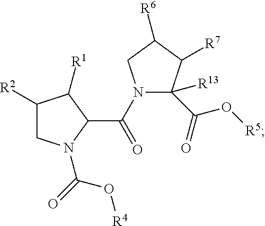
b) contacting the compound of Formula IV, or a salt thereof, with a reagent capable of effecting hydrolysis to produce a compound of Formula V, or a salt thereof: 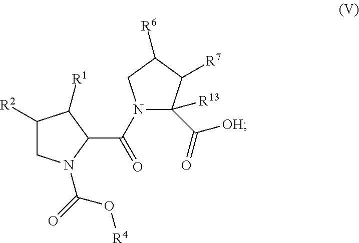
and
c) contacting the compound of Formula V, or a salt thereof, with an activating reagent and a compound of Formula VIII: 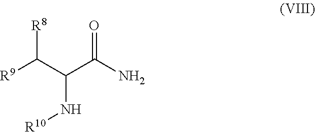
to produce a compound of Formula IX, or a salt thereof: 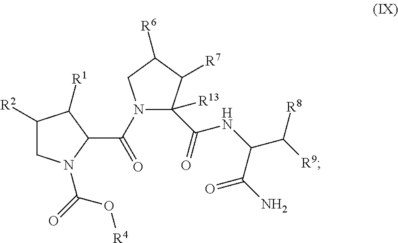
d) contacting the compound of Formula IX, or a salt thereof, with a carbamate-cleaving reagent to produce a compound of Formula XI, or a salt thereof: 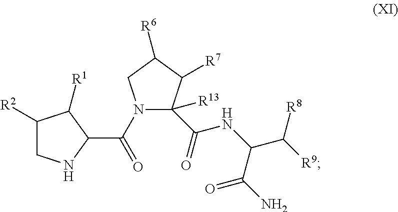
e) contacting a compound of Formula X, or a salt thereof: 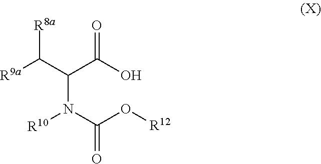
with an activating reagent and the compound of Formula XI, or a salt thereof, to produce a compound of Formula XIIa, or a salt thereof: 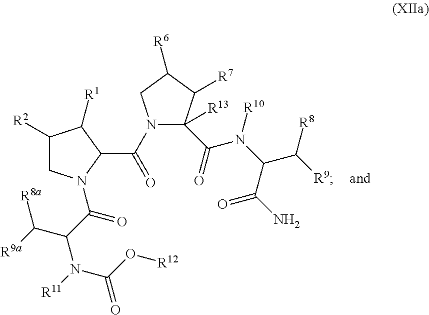
and
f) contacting the compound of Formula XIIa, or a salt thereof, with a silyl ether cleaving reagent to produce a compound of Formula XII, or a salt thereof: 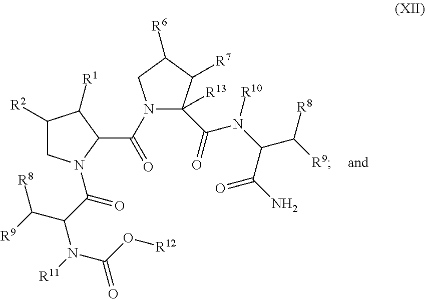
and
g) contacting the compound of Formula XII, or a salt thereof, with a carbamate-cleaving reagent to produce a compound of Formula XIII, or a salt thereof: 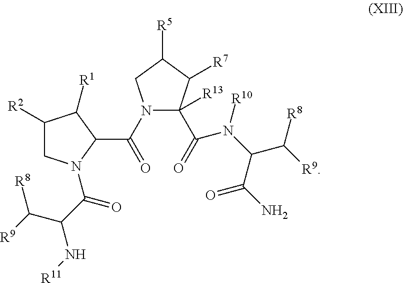
wherein:
R¹ and R² are independently selected from the group consisting of hydrogen; halogen; hydroxyl; substituted or unsubstituted C₁₋₆alkyl; substituted or unsubstituted C₁₋₆alkoxy; and substituted or unsubstituted aryl; or R¹ and R², together with the atoms to which they are attached, form a substituted or unsubstituted 4-6 membered heterocyclic or cycloalkyl ring;
R⁴, R⁵, and R¹² are independently —C₁₋₆alkylene-phenyl, wherein C₁₋₆alkylene is optionally substituted by one or more substituents each independently selected from R^{f};
R⁶ and R⁷ are independently selected from the group consisting of hydrogen; halogen; hydroxyl; substituted or unsubstituted C₁₋₆alkyl; substituted or unsubstituted C₁₋₆alkoxy; and substituted or unsubstituted aryl; or R⁶ and R⁷, together with the atoms to which they are attached, form a substituted or unsubstituted 4-6 membered heterocyclic or cycloalkyl ring;
R⁸ and R⁹ are independently selected from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkylene-; naphthyl-C₁₋₆alkylene-; heteroaryl-C₁₋₆alkylene-; and heterocyclyl-C₁₋₆alkylene-; —OR^{x}; —NO₂; —N₃; —CN; —SCN; —SR^{x}; —C(O)R⁸; —CO₂(R^{x}); —C(O)N(R^{x})₂; —C(NR^{x})N(R^{x})₂; —OC(O)R^{x}; —OCO₂R^{x}; —OC(O)N(R^{x})₂; —N(R^{x})₂; —SOR^{x}; —S(O)₂R^{x}; —NR^{x}C(O)R^{x}; —NR^{x}C(O)N(R^{x})₂; —NR^{x}C(O)OR^{x}; —NR^{x}C(NR^{x})N(R^{x})₂; and —C(R^{x})₃; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl are each independently optionally substituted by one or more substituents each independently selected from R^{e}; wherein C₁₋₆alkyl and C₁₋₆alkylene are each independently optionally substituted by one or more substituents each independently selected from R^{f}; wherein C₃₋₆cycloalkyl is independently optionally substituted by one or more substituents each independently selected from R^{g};
R^{8a} and R^{9a} may be independently selected from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkylene-; naphthyl-C₁₋₆alkylene-; heteroaryl-C₁₋₆alkylene-; and heterocyclyl-C₁₋₆alkylene-; —OR^{x}; —OR^{y}, —NO₂; —N₃; —CN; —SCN; —SR^{x}; —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —N(R^{x})₂; —SOR^{x}; —S(O)₂R^{x}; —NR^{x}C(O)R^{x}; —NR^{x}C(O)N(R^{x})₂; —NR^{x}C(O)OR^{x}; —NR^{x}C(NR^{x})N(R^{x})₂; and —C(R^{x})₃; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl are each independently optionally substituted by one or more substituents each independently selected from R^{e}; wherein C₁₋₆alkyl and C₁₋₆alkylene are each independently optionally substituted by one or more substituents each independently selected from R^{f}; wherein C₃₋₆cycloalkyl is independently optionally substituted by one or more substituents each independently selected from R^{g}; wherein one of R^{8a} or R^{9a} is —OR^{y}.
R¹⁰ and R¹¹ are independently selected from the group consisting of hydrogen; C₁₋₆alkyl; —C(O)—C₁₋₆alkylene; —C(O)—O—C₁₋₆alkylene; and —C(O)-phenyl; wherein C₁₋₆alkyl, C₁₋₆alkylene, and phenyl are optionally independently substituted by one or more substituents selected from R^{a};
R¹³ is selected from hydrogen or benzyl;
R^{b} is selected, independently for each occurrence, from the group consisting of halogen; hydroxyl; —NO₂; —N₃; —CN; —SCN; C₁₋₆alkyl; C₂₋₆alkenyl; C₂₋₆alkynyl; C₃₋₆cycloalkyl; C₁₋₆alkoxy; C₃₋₆alkenyloxy; C₃₋₆alkynyloxy; C₃₋₆cycloalkoxy; C₁₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;
R^{a} and R^{a′} are selected, independently for each occurrence, from the group consisting of hydrogen and C₁₋₆alkyl, or R^{a} and R^{a′} when taken together with the nitrogen to which they are attached form a 4-6 membered heterocyclic ring, wherein C₁₋₆alkyl is optionally substituted by one or more substituents each independently selected from the group consisting of halogen, oxo, and hydroxyl, and wherein the heterocyclic ring is optionally substituted by one or more substituents each independently selected from the group consisting of halogen, alkyl, oxo, or hydroxyl;
R^{c} is selected, independently for each occurrence, from the group consisting of halogen; hydroxyl; —NO₂; —N₃; —CN; —SCN; oxo; C₁₋₆alkyl; C₂₋₆alkenyl; C₂₋₆alkynyl; C₃₋₆cycloalkyl; C₁₋₆alkoxy; C₃₋₆alkenyloxy; C₃₋₆alkynyloxy; C₃₋₆cycloalkoxy; C₁₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N—; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;
R^{d} is selected, independently for each occurrence, from the group consisting of C₁₋₆alkyl, C₁₋₆alkylcarbonyl, and C₁₋₆alkylsulfonyl, wherein C₁₋₆alkyl is optionally substituted by one or more substituents each independently selected from halogen, hydroxyl, and R^{a}R^{a′}N—;
R^{e} is selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{f} is selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{g} is selected, independently for each occurrence, from the group consisting of halogen, hydroxyl, —NO₂; —N₃; —CN; —SCN; C₁₋₆alkyl; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{x} is selected, independently, from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkyl-; naphthyl-C₁₋₆alkyl-; heteroaryl-C₁₋₆alkyl-; and heterocyclyl-C₁₋₆alkyl-; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl, are each independently optionally substituted by one or more substituents each independently selected from R^{e}; wherein C₁₋₆alkyl is optionally substituted by one or more substituents each independently selected from R^{f}; wherein C₃₋₆cycloalkyl is independently optionally substituted by one or more substituents each independently selected from R^{g}; and
R^{y} is selected, independently, from the group consisting of alkylsilyl, arylsilyl and heteroarylsilyl, wherein the heteroaryl of heteroarylsilyl is a 5-6 membered ring having one, two, or three heteroatoms each independently selected from N, O, or S and optionally substituted with one or more substituents each independently selected from R^{b}; wherein the alkyl or aryl of the alkylsilyl or arylsilyl, respectively, is optionally substituted by one or more substituents each independently selected from R^{f}.

2. The process of claim 1, wherein the compound of Formula X, or a salt thereof, is produced by (x) contacting a compound of Formula VI, or a salt thereof: 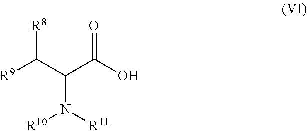
with an activated carbonyl compound to produce a compound of Formula (Xa), or a salt thereof: 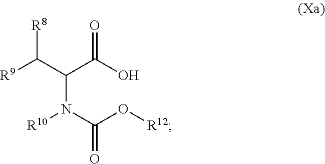
and
(y) contacting a compound of Formula X, or a salt thereof, with a silyl halide to produce a compound of Formula (X), or a salt thereof:

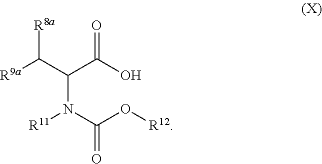

3. The process of claim 1, wherein the compound of Formula VIII, or a salt thereof, is produced by the steps:
g) contacting a compound represented by Formula VI, or a salt thereof: 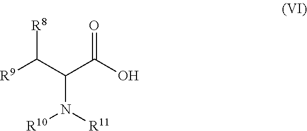
with an activating reagent to form a compound represented by Formula VII, or a salt thereof: 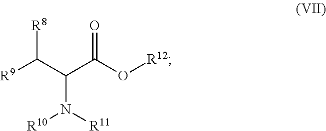
and
h) contacting the compound of Formula VII, or a salt thereof with an amine to produce the compound of Formula VIII, or a salt thereof.

4. The process according to claim 1, wherein the compound of Formula II, or a salt thereof, is produced by contacting a compound of Formula I, or a salt thereof: 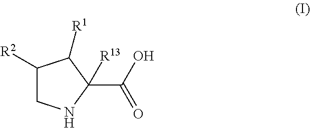
with an activating reagent and an alcohol.

5. The process according to claim 1, wherein the compound of Formula III, or a salt thereof, is produced by contacting the compound of Formula II, or a salt thereof, with an activated carbonyl reagent and a base.

6. The process of claim 3, wherein the activating reagent comprises SOCl₂.

7. The process of claim 3, wherein the alcohol is MeOH.

8. The process of claim 5, wherein the activated carbonyl reagent is Cbz-Cl.

9. The process of claim 5, wherein the base is a hydroxide salt.

10. (canceled)

11. The process according to claim 1, wherein the reagent capable of effecting hydrolysis of the compound of Formula IV, or a salt thereof, comprises LiOH.

12. (canceled)

13. The process of claim 3, wherein the amine is NH₃.

14. The process according to claim 1, wherein the activating reagent in step (c) comprises 1-ethyl-3-(3-dimethyllaminopropyl)carbodiimide.

15. The process of claim 2, wherein the activated carbonyl compound is Cbz-Cl.

16. The process of claim 2, further comprising contacting the compound of Formula VI, or a salt thereof, with a base.

17. The process of claim 16, wherein the base is NaHCO₃.

18. The process according to claim 1, wherein the carbamate-cleaving reagent comprises palladium on carbon.

19. The process of claim 1, wherein the activating reagent comprises 1-ethyl-3-(3-dimethyllaminopropyl)carbodiimide.

20. A process for preparing a dipyrrolidine peptide compound or a pharmaceutically acceptable salt, stereoisomer, metabolite, or hydrate thereof, comprising the steps:
a) contacting a compound of Formula IX, or a salt thereof: 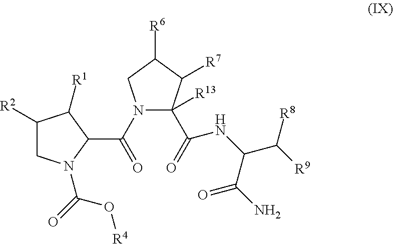
with a carbamate-cleaving reagent to produce a compound of Formula XI, or a salt thereof: 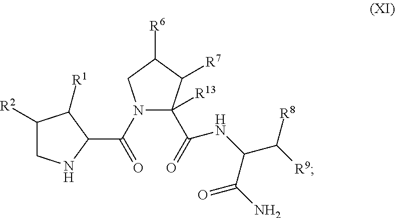
b) contacting a compound of Formula X, or a salt thereof: 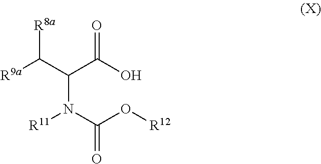
with an activating reagent and the compound of Formula XI, or a salt thereof, in the presence of at least one solvent to produce a compound of Formula XIIa, or a salt thereof: 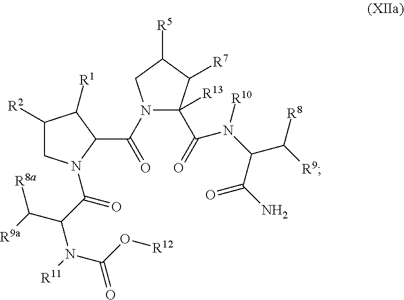
and
c) contacting the compound of Formula XIIa, or a salt thereof, with a silyl ether cleaving reagent to produce a compound of Formula XII, or a salt thereof: 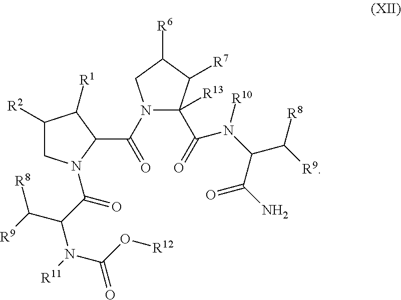
and
d) contacting the compound of Formula XII, or a salt thereof, with a carbamate-cleaving reagent to produce a compound of Formula XIII, or a salt thereof: 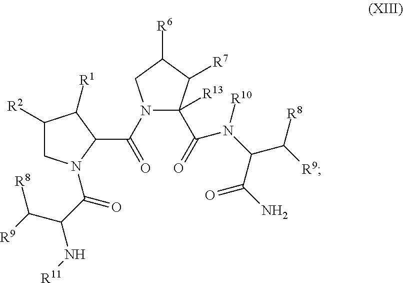
wherein:
R¹ and R² may be independently selected from the group consisting of hydrogen; halogen; hydroxyl; substituted or unsubstituted C₁₋₆alkyl; substituted or unsubstituted C₁₋₆alkoxy; and substituted or unsubstituted aryl; or R¹ and R², together with the atoms to which they are attached, form a substituted or unsubstituted 4-6 membered heterocyclic or cycloalkyl ring;
R⁴, R⁵, and R¹² may be independently —C₁₋₆alkylene-phenyl, wherein C₁₋₆alkylene is optionally substituted by one or more substituents each independently selected from R^{f};
R⁶ and R⁷ may be independently selected from the group consisting of hydrogen; halogen; hydroxyl; substituted or unsubstituted C₁₋₆alkyl; substituted or unsubstituted C₁₋₆alkoxy; and substituted or unsubstituted aryl; or R⁶ and R⁷, together with the atoms to which they are attached, form a substituted or unsubstituted 4-6 membered heterocyclic or cycloalkyl ring;
R⁸ and R⁹ may be independently selected from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkylene-; naphthyl-C₁₋₆alkylene-; heteroaryl-C₁₋₆alkylene-; and heterocyclyl-C₁₋₆alkylene-; —OR^{x}; —NO₂; —N₃; —CN; —SCN; —SR^{x}; —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —N(R^{x})₂; —SOR^{x}; —S(O)₂R^{x}; —NR^{x}C(O)R^{x}; —NR^{x}C(O)N(R^{x})₂; —NR^{x}C(O)OR^{x}; —NR^{x}C(NR^{x})N(R^{x})₂; and —C(R^{x})₃; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl are each independently optionally substituted by one or more substituents each independently selected from R^{e}; wherein C₁₋₆alkyl and C₁₋₆alkylene are each independently optionally substituted by one or more substituents each independently selected from R^{f}; wherein C₃₋₆cycloalkyl is independently optionally substituted by one or more substituents each independently selected from R^{g};
R^{8a} and R^{9a} may be independently selected from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkylene-; naphthyl-C₁₋₆alkylene-; heteroaryl-C₁₋₆alkylene-; and heterocyclyl-C₁₋₆alkylene-; —OR^{x}; —OR^{y}, —NO₂; —N₃; —CN; —SCN; —SR^{x}; —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —N(R^{x})₂; —SOR^{x}; —S(O)₂R^{x}; —NR^{x}C(O)R^{x}; —NR^{x}C(O)N(R^{x})₂; —NR^{x}C(O)OR^{x}; —NR^{x}C(NR^{x})N(R^{x})₂; and —C(R^{x})₃; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl are each independently optionally substituted by one or more substituents each independently selected from R^{e}; wherein C₁₋₆alkyl and C₁₋₆alkylene are each independently optionally substituted by one or more substituents each independently selected from R^{f}; wherein C₃₋₆cycloalkyl is independently optionally substituted by one or more substituents each independently selected from R^{g}; wherein one of R^{8a} or R^{9a} is —OR^{y};
R¹⁰ and R¹¹ are independently selected from the group consisting of hydrogen; C₁₋₆alkyl; —C(O)—C₁₋₆alkylene; —C(O)—O—C₁₋₆alkylene; and —C(O)-phenyl; wherein C₁₋₆alkyl, C₁₋₆alkylene, and phenyl are optionally independently substituted by one or more substituents selected from R^{a};
R¹³ is selected from hydrogen or benzyl;
R^{b} may be selected, independently for each occurrence, from the group consisting of halogen; hydroxyl; —NO₂; —N₃; —CN; —SCN; C₁₋₆alkyl; C₂₋₆alkenyl; C₂₋₆alkynyl; C₃₋₆cycloalkyl; C₃₋₆alkoxy; C₃₋₆alkenyloxy; C₃₋₆alkynyloxy; C₃₋₆cycloalkoxy; C₃₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{x})—; C₁₋₆alkylN(R^{a})—; C₁₋₆alkyl-N(R^{x})carbonyl-; R^{a}R^{a′}N—; R^{a}R^{a′}N-carbonyl-; R^{a}R^{a′}N-carbonyl-N(R^{x})—; R^{a}R^{a′}N—SO₂—; and C₁₋₆alkyl-carbonyl-N(R^{a})—;
R^{a} and R^{a′} may be selected, independently for each occurrence, from the group consisting of hydrogen and C₁₋₆alkyl, or R^{a} and R^{a′} when taken together with the nitrogen to which they are attached form a 4-6 membered heterocyclic ring, wherein C₁₋₆alkyl is optionally substituted by one or more substituents each independently selected from the group consisting of halogen, oxo, and hydroxyl, and wherein the heterocyclic ring is optionally substituted by one or more substituents each independently selected from the group consisting of halogen, alkyl, oxo, or hydroxyl;
R^{c} may be selected, independently for each occurrence, from the group consisting of halogen; hydroxyl; —NO₂; —N₃; —CN; —SCN; oxo; C₁₋₆alkyl; C₂₋₆alkenyl; C₂₋₆alkynyl; C₃₋₆cycloalkyl; C₃₋₆alkoxy; C₃₋₆alkenyloxy; C₃₋₆alkynyloxy; C₃₋₆cycloalkoxy; C₃₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N—; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;
R^{d} may be selected, independently for each occurrence, from the group consisting of C₁₋₆alkyl, C₁₋₆alkylcarbonyl, and C₁₋₆alkylsulfonyl, wherein C₁₋₆alkyl is optionally substituted by one or more substituents each independently selected from halogen, hydroxyl, and R^{a}R^{a′}N—;
R^{e} may be selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{f} may be selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{g} may be selected, independently for each occurrence, from the group consisting of halogen, hydroxyl, —NO₂; —N₃; —CN; —SCN; C₁₋₆alkyl; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{x} is selected, independently, from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkyl-; naphthyl-C₁₋₆alkyl-; heteroaryl-C₁₋₆alkyl-; and heterocyclyl-C₁₋₆alkyl-; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl, are each independently optionally substituted by one or more substituents each independently selected from R^{e}; wherein C₁₋₆alkyl is optionally substituted by one or more substituents each independently selected from R^{f}; wherein C₃₋₆cycloalkyl is independently optionally substituted by one or more substituents each independently selected from R^{g}; and
R^{y} is selected, independently, from the group consisting of alkylsilyl, arylsilyl and heteroarylsilyl, wherein the heteroaryl of heteroarylsilyl is a 5-6 membered ring having one, two, or three heteroatoms each independently selected from N, O, or S and optionally substituted with one or more substituents each independently selected from R^{b}; wherein the alkyl or aryl of the alkylsilyl or arylsilyl, respectively, is optionally substituted by one or more substituents each independently selected from R^{f}.

21-48. (canceled)

49. The process according to claim 8, wherein the compound of Formula I is:

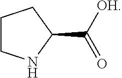

50. The process according to claim 49, wherein the compound of Formula II is:

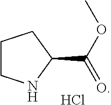

51. The process according to claim 50, wherein the compound of Formula III is:

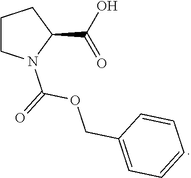 [SMILES: O=C(O)[C@@H]1CCCN1C(=O)OCc1ccccc1]

52. The process according to claim 51, wherein the compound of Formula IV is:

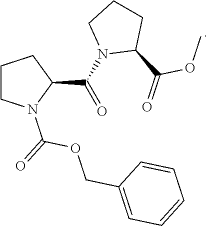

53. The process according to claim 52, wherein the compound of Formula V is:

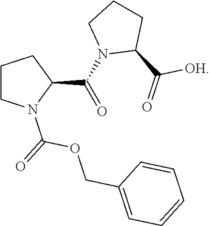

54. The process according to claim 53, wherein the compound of Formula VI is:

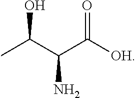

55. The process according to claim 54, wherein the compound of Formula VII is:

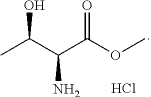

56. The process according to claim 55, wherein the compound of Formula VIII is:

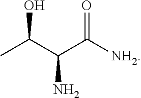

57. The process according to claim 56, wherein the compound of Formula IX is:

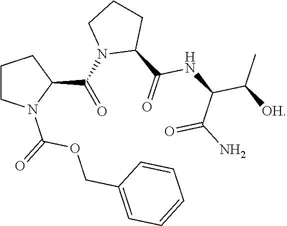

58. The process according to claim 57, wherein the compound of Formula X is:

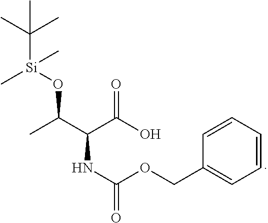 [SMILES: C[C@@H](O[Si](C)(C)C(C)(C)C)[C@H](NC(=O)OCc1ccccc1)C(=O)O]

59. The process according to claim 58, wherein the compound of Formula XI is:

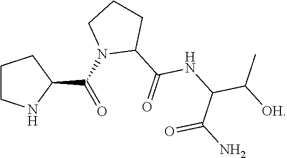

60. The process according to claim 59, wherein the compound of Formula XIIa is:

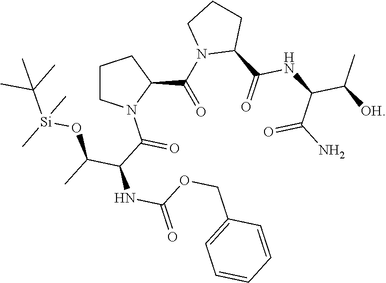 [SMILES: C[C@@H](O)[C@H](NC(=O)[C@@H]1CCCN1C(=O)[C@@H]1CCCN1C(=O)[C@@H](NC(=O)OCc1ccccc1)[C@@H](C)O[Si](C)(C)C(C)(C)C)C(N)=O]

61. The process according to claim 60, wherein the compound of Formula XIII is:

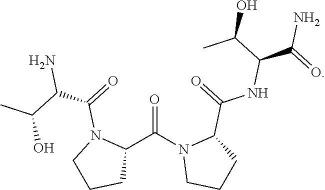

62. A compound represented by the Formula X, or a salt thereof: 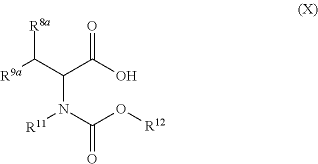
wherein:
R^{8a} and R^{9a} may be independently selected from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkylene-; naphthyl-C₁₋₆alkylene-; heteroaryl-C₁₋₆alkylene-; and heterocyclyl-C₁₋₆alkylene-; —OR^{x}; —OR^{y}, —NO₂; —N₃; —CN; —SCN; —SR^{x}; —C(O)R^{x}; —CO₂(R^{x}); —C(O)N(R^{x})₂; —C(NR^{x})N(R^{x})₂; —OC(O)R^{x}; —OCO₂R^{x}; —OC(O)N(R^{x})₂; —N(R^{x})₂; —SOR^{x}; —S(O)₂R^{x}; —NR^{x}C(O)R^{x}; —NR^{x}C(O)N(R^{x})₂; —NR^{x}C(O)OR^{x}; —NR^{x}C(NR^{x})N(R^{x})₂; and —C(R^{x})₃; wherein heteroaryl is a 5-6 membered ring having one, two, or three heteroatoms each independently selected from N, O, or S; wherein heteroaryl is optionally substituted with one or more substituents each independently selected from R^{b}); wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl are each independently optionally substituted by one or more substituents each independently selected from R^{e}; wherein C₁₋₆alkyl and C₁₋₆alkylene are each independently optionally substituted by one or more substituents each independently selected from R^{f}; wherein C₃₋₆cycloalkyl is independently optionally substituted by one or more substituents each independently selected from R^{g}; wherein one of R^{8a} or R^{9a} is —OR^{y};
R¹¹ is selected from the group consisting of hydrogen; C₁₋₆alkyl; —C(O)—C₁₋₆alkylene; —C(O)—O—C₁₋₆alkylene; and —C(O)-phenyl; wherein C₁₋₆alkyl, C₁₋₆alkylene, and phenyl are optionally independently substituted by one or more substituents selected from R^{a};
R^{b} is selected, independently for each occurrence, from the group consisting of halogen; hydroxyl; —NO₂; —N₃; —CN; —SCN; C₁₋₆alkyl; C₂₋₆alkenyl; C₂₋₆alkynyl; C₃₋₆cycloalkyl; C₃₋₆alkoxy; C₃₋₆alkenyloxy; C₃₋₆alkynyloxy; C₃₋₆cycloalkoxy; C₁₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N—; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;
R^{a} and R^{a′} is selected, independently for each occurrence, from the group consisting of hydrogen and C₁₋₆alkyl, or R^{a} and R^{a′} when taken together with the nitrogen to which they are attached form a 4-6 membered heterocyclic ring, wherein C₁₋₆alkyl is optionally substituted by one or more substituents each independently selected from the group consisting of halogen, oxo, and hydroxyl, and wherein the heterocyclic ring is optionally substituted by one or more substituents each independently selected from the group consisting of halogen, alkyl, oxo, or hydroxyl;
R^{c} is selected, independently for each occurrence, from the group consisting of halogen; hydroxyl; —NO₂; —N₃; —CN; —SCN; oxo; C₁₋₆alkyl; C₂₋₆alkenyl; C₂₋₆alkynyl; C₃₋₆cycloalkyl; C₁₋₆alkoxy; C₃₋₆alkenyloxy; C₃₋₆alkynyloxy; C₃₋₆cycloalkoxy; C₃₋₆alkyl-S(O)_{w}—, where w is 0, 1, or 2; C₁₋₆alkylC₃₋₆cycloalkyl-; C₃₋₆cycloalkyl-C₁₋₆alkyl-; C₁₋₆alkoxycarbonyl-N(R^{a})—; C₁₋₆alkylN(R^{a})—; C₁₋₆alkyl-N(R^{a})carbonyl-; R^{a}R^{a′}N-carbonyl-; R^{a}R^{a′}N-carbonyl-N(R^{a})—; R^{a}R^{a′}N—SO₂—; and C₁₋₆alkyl-carbonyl-N(R^{a})—;
R^{d} is selected, independently for each occurrence, from the group consisting of C₁₋₆alkyl, C₁₋₆alkylcarbonyl, and C₁₋₆alkylsulfonyl, wherein C₁₋₆alkyl is optionally substituted by one or more substituents each independently selected from halogen, hydroxyl, and R^{a}R^{a′}N—;
R^{e} is selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{f} is selected, independently for each occurrence, from the group consisting of halogen; hydroxyl; —NO₂; —N₃; —CN; —SCN; C₁₋₄alkoxy; C₁₋₄alkoxycarbonyl; R^{a}R^{a′}N-carbonyl; R^{a}R^{a′}N—SO₂—; and C₁₋₄alkylS(O)_{w}—, where w is 0, 1, or 2;
R^{g} is selected, independently for each occurrence, from the group consisting of halogen, hydroxyl, —NO₂; —N₃; —CN; —SCN; C₁₋₆alkyl; C₁₋₄alkoxy; C₁₋₄alkoxycarbonyl; R^{a}R^{a′}N—; R^{a}R^{a′}N-carbonyl; R^{a}R^{a′}N—SO₂—; and C₁₋₄alkylS(O)_{w}—, where w is 0, 1, or 2;
R^{x} is selected, independently, from the group consisting of hydrogen; halogen; C₁₋₆alkyl; C₂₋₆alkenyl; C₂₋₆alkynyl; C₃₋₆cycloalkyl; phenyl; naphthyl; heteroaryl; heterocyclyl; C₃₋₆cycloalkyl-C₁₋₆alkyl-; phenyl-C₁₋₆alkyl-; naphthyl-C₁₋₆alkyl-; heteroaryl-C₁₋₆alkyl-; and heterocyclyl-C₁₋₆alkyl-; wherein heteroaryl is a 5-6 membered ring having one, two, or three heteroatoms each independently selected from N, O, or S; wherein heteroaryl is optionally substituted with one or more substituents each independently selected from R^{b}; wherein heterocyclyl is a 4-7 membered ring optionally substituted by one or more substituents each independently selected from R^{c}; wherein when heterocyclyl contains a —NH— moiety, that —NH— moiety is optionally substituted by R^{d}; wherein C₂₋₆alkenyl and C₂₋₆alkynyl, are each independently optionally substituted by one or more substituents each independently selected from R^{e}; wherein C₁₋₆alkyl is optionally substituted by one or more substituents each independently selected from R^{f}; wherein C₃₋₆cycloalkyl is independently optionally substituted by one or more substituents each independently selected from R^{g}; and
R^{y} is selected, independently, from the group consisting of alkylsilyl, arylsilyl and heteroarylsilyl, wherein the heteroaryl of heteroarylsilyl is a 5-6 membered ring having one, two, or three heteroatoms each independently selected from N, O, or S and optionally substituted with one or more substituents each independently selected from R^{b}; wherein the alkyl or aryl of the alkylsilyl or arylsilyl, respectively, is optionally substituted by one or more substituents each independently selected from R^{f}.

63-66. (canceled)

67. The compound of claim 62, represented by the formula:

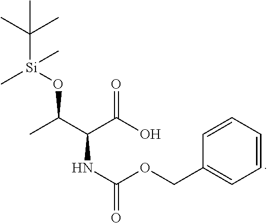 [SMILES: C[C@@H](O[Si](C)(C)C(C)(C)C)[C@H](NC(=O)OCc1ccccc1)C(=O)O]

68. The compound represented by the formula:

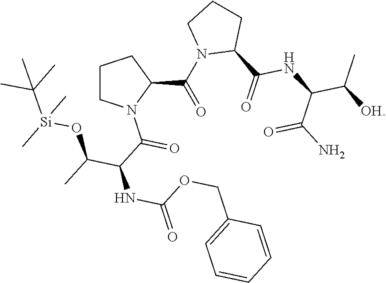 [SMILES: C[C@@H](O)[C@H](NC(=O)[C@@H]1CCCN1C(=O)[C@@H]1CCCN1C(=O)[C@@H](NC(=O)OCc1ccccc1)[C@@H](C)O[Si](C)(C)C(C)(C)C)C(N)=O]

69-73. (canceled)

74. A compound selected from: 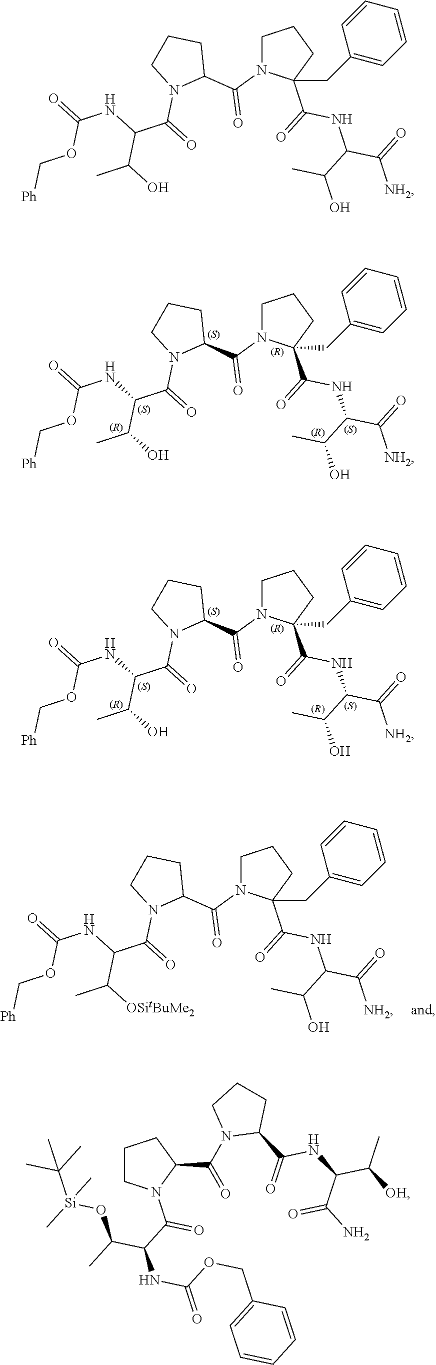
or a salt thereof.

75-79. (canceled)
